(19) United States
(12) Patent Application Publication
(10) Pub. No.: US 2007/0269790 A1
(43) Pub. Date: Nov. 22, 2007

(54) Methods of Generating Stem Cells and Embryonic Bodies Carrying Disease-Causing Mutations and Methods of Using same for Studying Genetic Disorders

(75) Inventors: Michal Amit, Misgav (IL); Joseph Itskovitz-Eldor, Haifa (IL)

Correspondence Address: Martin D. Moynihan;PRTSI, Inc., P.O. Box 16446, Arlington, VA, 22215, US

(73) Assignee: TECHNION RESEARCH & DEVELOPMENT, HAIFA (IL)

(21) Appl. No.: 10/581,455

(22) PCT Filed:
(86) PCT No.: PCT/IL04/01046

Related U.S. Application Data
(60) Provisional application No. 60/525,883, filed on Dec. 1, 2003.

(51) Int. Cl. C12N 5/08; A01N 1/02

(52) U.S. Cl. 435/1.1; 435/29; 435/325; 435/368

(57) ABSTRACT

Stem cells, stem cell lines and differentiated cells, tissues and organs which carry disease-causing mutations are provided. There is also provided a method of identifying agents suitable for treating disorders associated with at least one disease-causing mutations such as myotonic dystrophy and van Waardenburg syndrome.

### FIELD AND BACKGROUND OF THE INVENTION

The present invention relates to human embryonic stem (ES) cells which carry disease-causing mutations, and more particularly, to methods of using such cells in developing treatment for genetic disorders such as myotonic dystrophy and van Waardenburg syndrome.

Genetic disorders result from chromosomal aberrations such as trisomies, monosomies, deletions, duplications and inversions, and/or from DNA abnormalities such as single nucleotide substitutions, deletion, insertion, or repeat expansion in one or more genes. Such chromosomal and/or DNA abnormalities are often transmitted in a recessive (e.g., cystic fibrosis and Canavan), dominant (e.g., Myotonic Dystrophy) or imprinting (e.g., Prader-Willi or Angelman syndromes) mode of inheritance.

For example, myotonic dystrophy (DM1) or Steinert's disease is an autosomal dominant, late-onset, myotonic disorder affecting 2.1-14.3 out of 100,000 live-birth individuals worldwide (Meola, 2000). DM is characterized by progressive muscle wasting, cataract, nervous system dysfunction, cardiac conduction abnormalities and endocrine abnormalities such as diabetes and gonadal atrophy (Mankodi and Thornton, 2002). DM1 results from abnormal expansions of a (CTG)ₙ repeat in the 3′-untranslated region (3′-UTR) of the DMPK gene (GenBank Accession No. NM_{—}004409). Thus, while normal individuals exhibit between 5-30 repeat copies, mildly affected individuals exhibit 50-80 repeat copies and severely affected individuals exhibit more than 2,000 copies (Brook et al, 1992).

Other examples of autosomal dominant disorders include the Van Waardenburg syndrome (WS1, Waardenburg, 1951) and Huntington's disease (HD). Van Waardenburg syndrome is characterized by a wide bridge of the nose owing to lateral displacement of the inner canthus of each eye, pigmentary disturbance (frontal white blaze of hair, heterochromia iridis, white eye lashes, leukoderma), and cochlear deafness (McKusick 1992; Waardenburg, 1951). The incidence prevalence of the disease is estimated to be between 1.44 to 2.05 newborns out of 100,000 deliveries worldwide (Fraser, 1976). Deletion of the whole PAX3 gene (GenBank Accession No. NM 000438) or single-base substitutions in the paired domain or the homeodomain of PAX3 were found to cause WS1 (Baldwin et al, 1992; Tassabehji et al, 1992). Huntington's disease (HD) is characterized by a progressive, localized neural cell death which leads to choreic movements and dementia. The disease is associated with increases in the length of a CAG triplet repeat present in a gene called ‘huntingtin’ located on chromosome 4p16.3.

Cystic fibrosis (CF) is an autosomal recessive disorder characterized by disruptions of the exocrine function of the pancreas, intestinal glands, biliary tree, bronchial glands, and sweat glands. CF is caused by mutations in the cystic fibrosis conductance regulator (CFTR) gene (GenBank Accession No. M28668, Kerem, B., et al., 1989, Science 245: 1073-1080) and its estimated incidence in the USA is 1 out of 3419 live-birth among the white population, and 1 out of 12,163 live-birth among the other populations (Kosorok M R, et al., 1996, Stat. Med. 15: 449-62).

Another example of an autosomal recessive disorder is the lysosomal storage metachromatic leukodystrophy (MLD) disorder. MLD results from mutations in two different genes, arylsulfatase A (ARSA, GenBank Accession No. AY271820) and prosaposin (GenBank Accession No. BT006849), both of which encode for proteins needed for proper degradation of cerebroside sulfate, a glycolipid mainly found in the myelin membranes (Gieselmann V, et al., 1994, Hum. Mutat. 4: 233-42).

Still another example of an autosomal recessive disease is spinal muscular atrophy (SMA) which is caused by disruption of the telomeric copy of a duplicated gene called survival motor neuron (SMN1). SMA is characterized by degeneration of the anterior horn cells leading to symmetrical muscle weakness and wasting of voluntary muscles.

Duchenne muscular dystrophy (DMD) is an X-linked genetic disease caused by mutation in the gene encoding dystrophin and characterized by a progressive proximal muscular dystrophy with characteristic pseudohypertrophy of the calves. The disease affects a wide variety of tissues including, skeletal muscle, cardiac muscle, smooth muscle, nervous system, retina and myoblasts.

However, although many of such genetic disorders can be diagnosed prenatally (using chorionic villi or amniotic fluid samples), or even prior to the implantation of an in vitro fertilized embryo (at the blastocyst stage) in the uterus, in most cases, the processes leading to the overall disorder's phenotype are unknown.

To farther understand the molecular and physiological basis of such disorders and in attempts to develop proper treatments, several disease-models, such as cell cultures and animal models, have been constructed. Examples include the splotch-delayed (Spd) mouse mutant which carries a point mutation in the Pax-3 gene (Vogan K J, et al., 1993, Genomics. 17: 364-9; Asher et al, 1996) as a model for WS; the DMPK-deficient mice (Berul C I, et al., 2000, J. Interv. Card. Electrophysiol. 4: 351-8) and the C2C12 mouse myoblast cells expressing chimeric reporter gene fused to a human DMPK 3′-UTR (Amack J D, et al., 1999, Hum. Mol. Genet. 8: 1975-84) as models for DM1; the CF-mouse models [e.g., delta-F508 (van Doorninck J H, et al., 1995, EMBO J. 14: 4403-11) and G480C (Dickinson P et al., 2002, Mol. Genet. 11: 243-51)]; and the arylsulfatase A-deficient mice (D'Hooge R, et al., 2001, Brain Res. 907: 35-43) as a model for MLD. However, although such disease-models present biochemical models of the disorder, they often do not reproduce the clinical symptoms (Elsea S H, Lucas R E., 2002, ILAR J. 43: 66-79), probably as a result of various cloning artifacts and differences in the genetic make-up between various species (i.e., mouse and human). Thus, the presently available disease-models are not suitable for developing cures for genetic disorders.

Embryonic stem (ES) cells are pluripotent stem cells which are capable of prolonged undifferentiated proliferation while maintaining normal karyotype, as well as differentiation into cells of all embryonic germ layers, i.e., the endoderm, ectoderm and mesoderm and developing into all types of cells, tissues, organs and/or body parts, including a whole organism. Thus, ES cells may be used to study the mechanisms leading to developmental and differentiation processes, lineage commitment, self-maintenance and maturation of progenitor cells. Moreover, ES cells can be used in cell-based therapy and regeneration of many genetic and acquired diseases such as Parkinson's disease, cardiac infarcts, juvenile-onset diabetes mellitus, and leukemia (Gearhart J. Science 1998, 282:1061; Rossant and Nagy, Nature Biotech. 1999, 17:23).

While reducing the present invention to practice the present inventors have uncovered that embryos carrying naturally occurring disease-causing mutations can be used to generate ES cell lines and that such ES cell lines can be further differentiated to various experimental models of the genetic disorders associated with the disease-causing mutations.

### SUMMARY OF THE INVENTION

According to one aspect of the present invention there is provided an isolated stem cell or stem cell line carrying a disease-causing mutation in a genomic polynucleotide sequence thereof.

According to another aspect of the present invention there is provided an isolated embryoid body comprising a plurality of cells at least some of which carry a disease-causing mutation in a genomic polynucleotide sequence thereof.

According to yet another aspect of the present invention there is provided an isolated differentiated cell, tissue or organ carrying at least one disease-causing mutation in a genomic polynucleotide sequence thereof.

According to still another aspect of the present invention there is provided a method of identifying an agent suitable for treating a disorder associated with at least one disease-causing mutation, comprising: (a) generating a stem cell line or an embryoid body carrying the at least one disease-causing mutation; (b) subjecting cells of the stem cell line or the embryoid body to differentiating conditions to thereby obtain differentiated cells exhibiting an effect of the at least one disease-causing mutation and; (c) exposing the differentiated cells to a plurality of molecules and identifying from the plurality of molecules at least one molecule capable of regulating the effect of the at least one disease-causing mutation on the differentiated cells, the at least one molecule being the agent suitable for treating the disorder associated with the at least one disease-causing-mutation.

According to still further features in the described preferred embodiments the stem cell is of embryonic origin.

According to still further features in the described preferred embodiments the stem cell is of human origin.

According to still further features in the described preferred embodiments the disease-causing mutation is selected from the group consisting of a missense mutation, a nonsense mutation, a frameshift mutation, a readthrough mutation, a promoter mutation, a regulatory mutation, a deletion, an insertion, an inversion, a splice mutation and a duplication.

According to still further features in the described preferred embodiments the disease-causing mutation is associated with a genetic disorder selected from the group consisting of cystic fibrosis (CF), myotonic dystrophy (DM), van Waardenburg syndrome (WS), metachromatic leukodystrophy (OLD), Gorlin disease, Huntington's disease (HD), spinal muscular atrophy (SMA) and Duchenne muscular dystrophy (DMD).

According to still further features in the described preferred embodiments the disease-causing mutation is selected from the group consisting of the W1282X as set forth in SEQ ID NO:24 associated with cystic fibrosis, the PAX3-de128 (510de128 in SEQ ID NO:34) associated with van Waardenburg syndrome, more than 50 (CTG) repeats as set forth in SEQ ID NO:22 associated with Myotonic dystrophy and the 1505C→T (P377L) as set forth in SEQ ID NO:21 associated with metachromatic leukodystrophy.

According to still further features in the described preferred embodiments the stem cell is capable of being maintained in an undifferentiated state for at least 41 passages.

According to still further features in the described preferred embodiments the stem cell exhibits a karyotype of 46, XX or 46, XY following at least 30 passages.

According to still further features in the described preferred embodiments the stem cell exhibits pluripotent capacity following 40 passages.

According to still further features in the described preferred embodiments the stem cell is suspended in a culture medium including serum or serum replacement.

According to still further features in the described preferred embodiments the serum is provided at a concentration of at least 10% and the serum replacement is provided at a concentration of at least 15%.

According to still further features in the described preferred embodiments the embryoid body is derived from a stem cell or a stem cell line.

According to still further features in the described preferred embodiments the embryoid body is capable of differentiating into cells of the embryonic ectoderm, embryonic endoderm and/or embryonic mesoderm.

According to still further features in the described preferred embodiments the cells of the embryonic ectoderm are selected from the group consisting of neural cells, retina cells and epidermal cells.

According to still further features in the described preferred embodiments the cells of the embryonic endoderm are selected from the group consisting of hepatocytes, pancreatic cells and secreting cells.

According to still further features in the described preferred embodiments the cells of the embryonic mesoderm are selected from the group consisting of osseous cells, cartilaginous cells, elastic cells, fibrous cells, myocytes, myocardial cells, bone marrow cells, endothelial cells, smooth muscle cells, and hematopoietic cells.

According to still further features in the described preferred embodiments the embryoid body is suspended in a culture medium including serum or serum replacement.

According to still further features in the described preferred embodiments the embryoid body is at least 1 day old.

According to still further features in the described preferred embodiments the differentiated cell is selected from the group consisting of neural cells, retina cells, epidermal cells, hepatocytes, pancreatic cells, osseous cells, cartilaginous cells, elastic cells, fibrous cells, myocytes, myocardial cells, bone marrow cells, endothelial cells, smooth muscle cells, and hematopoietic cells.

According to still further features in the described preferred embodiments the tissue is selected from the group consisting of brain tissue, retina, skin tissue, hepatic tissue, pancreatic tissue, bone, cartilage, connective tissue, muscle tissue, cardiac tissue brain tissue, vascular tissue, hematopoietic, fat tissue, renal tissue, pulmonary tissue, and gonadal tissue.

According to still further features in the described preferred embodiments the organ is selected from the group consisting of head, brain, eye, leg, hand, heart, stomach, liver kidney, lung, pancreas, ovary, and testis.

According to still further features in the described preferred embodiments the differentiated cell, tissue or organ is of human origin.

According to still further features in the described preferred embodiments the method further comprising a step of isolating lineage specific cells from the embryoid body prior to step (b).

According to still further features in the described preferred embodiments isolating lineage specific cells is effected by sorting of cells contained within the embryoid body via fluorescence activated cell sorter.

According to still further features in the described preferred embodiments isolating lineage specific cells is effected by a mechanical separation of cells, tissues and/or tissue-like structures contained within the embryoid body.

According to still further features in the described preferred embodiments the lineage specific cells are of the embryonic ectoderm and are selected from the group consisting of neural cells, retina cells and epidermal cells.

According to still further features in the described preferred embodiments the lineage specific cells are of the embryonic endoderm and are selected from the group consisting of hepatocytes, secretors cells and pancreatic cells.

According to still further features in the described preferred embodiments the lineage specific cells are of the embryonic mesoderm and are selected from the group consisting of osseous cells, cartilaginous cells, elastic cells, fibrous cells, myocytes, myocardial cells, bone marrow cells, endothelial cells, smooth muscle cells, and hematopoietic cells.

The present invention successfully addresses the shortcomings of the presently known configurations by providing a stem cell which carry a naturally occurring disease-causing mutation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d are micrographs illustrating the derivation of a human embryonic stem (ES) cell line. FIG. 1a—an expanded blastocyst (at day 6) derived from an embryo following PGD. Note that part of the trophoectoderm layer buds as a result of the drill performed in the zona pellucida. This embryo was used for the derivation of the I-5 (WS1) line. Size bar=30 μM; FIG. 1b—ICM outgrowth (marked by an arrow) of the I-7 (DM1) ES cell line six days post plating the whole embryo at the blastocyst stage on MEFs. Size bar=45 μM. FIG. 1c—a colony of the I-7 (DM1) cell line (at passage five) growing in the presence of MEFs. Size bar=45 μM; FIG. 1d—undifferentiated cells of the I-5 (SW1) ES cell line at passage 24. Note the typical spaces between the cells. Size bar=15 μM.

FIGS. 2a-b illustrate the presence of disease-causing mutations of the Van Waardenburg syndrome (WS) and Myotonic Dystrophy (DM) in human ES cell lines. FIG. 2a—Ethidium Bromide staining of an agarose gel depicting WS-specific PCR analysis; PCR was performed using the WS specific primers (SEQ ID NOs:5-8). Lane 1—WS-affected parent; lane 2—normal individual; lane 3—I-5 (WS1) ES cell line. Note the presence of two PCR products in the affected parent (lane 1) and the I-5 (WS1) ES cell line corresponding to the wild-type and the 28 bp-deleted alleles. FIG. 2b—Silver staining of DM-specific PCR products. PCR was performed using the DM specific primers (SEQ ID NOs:1-4). Lanes 1-3—PCR products of affected individuals; lane 4—PCR products of the I-7 (DM1) ES cell line; lanes 5-6—PCR products of normal individuals. Δ=The size of repeat expansion. Note that DM affected individuals exhibit high molecular weight bands due to an expansion of the (CTG)ₙ repeat unit by 1 kb (lane 1), 2.3 kb (lane 2) and 2.4 kb (lane 3) beyond the normal size. Also note the presence of the high molecular weight bands in the PCR product of the I-7 (DM1) ES cell line corresponding to expanded repeats of 1.4 and 3.0 kb beyond the normal size of the repeat unit.

FIGS. 3a-f are immunohistochemistry micrographs illustrating the expression of embryonic cell surface markers on the I-5 (WS1) ES cells following 44 passages. Shown are bright (FIGS. 3a, c, e) or dark (FIGS. 3b, d, f) field images of human I-5 (WS1) ES cells labeled with monoclonal antibodies specific to SSEA4 (FIGS. 3a-b), TRA-1-6 (FIGS. 3c-d), or TRA-1-81 (FIGS. 3e-f). Size bar=50 μM.

FIGS. 4a-f illustrate the differentiation of ES cell lines carrying disease-causing mutations into embryoid bodies (EBs). Shown are H&E staining of histological sections of EBs formed from the I-7 (DM1) (FIG. 4a, size bar=60 μM) or I-5 (WS1) (FIG. 4b, size bar—30 μM) ES cell lines, and representative immunohistochemistry staining of differentiating cells within the EBs derived from the DM1 and WS1 ES cell line using anti nestin (FIG. 4c, WS1), insulin (FIG. 4d, WS1) and troponin (FIGS. 4e and f, WS1 and DM1, respectively) antibodies. It is worth mentioning that EBs derived from both WS1 and DM1 lines expressed all of these genes, i.e., nestin, insulin and troponin. Size bar in FIGS. 4c-f=6 μM.

FIG. 5 illustrates RT-PCR determination of the differentiation stage of the I-7 (DM1) or the I-5 (WS1) ES cell lines and of the embryoid bodies (EBs) derived therefrom. Lane 1—I-7 (DM1) ES cell line grown for 34 passages; lane 2—the I-5 (WS1) ES cell line grown for 41 passages; lane 3—five-day-old EBs derived from the I-5 (WS1) ES cell line following 40 passages; lane 4—five-day-old EBs derived from the I-7 (DM1) ES cell line following 34 passages with the exception of EBs from passage 30 were used as a negative control to the OCT4 expression; The specificity of the reaction was verified in the absence of RNA (lane 5).

FIGS. 6a-d illustrate histological sections of teratomas derived from the I-7 (DM1) or the I-5 (WS1) ES cell lines. Teratoma sections include secretory epithelium rich in goblet cells and stratified epithelium (FIG. 6a, the I-5 (WS1) ESC line, size bar=60 μm), developing bone tissue containing developing bone marrow (FIG. 6b, the I-5 (WS1) ESC line, size bar=20 μm), developing bone tissue formed (FIG. 6c, the I-7 (DM1) ESC line, size bar=30 μm) and a developing eye-like structure and epithelium (FIG. 6d, the I-7 (DM1) ESC line, size bar=60 μm).

### DESCRIPTION OF THE PREFERRED EMBODIMENTS

The present invention is of a human embryonic stem (ES) cells which carry disease-causing mutations which can be used for generating differentiated cells, tissue, embryoid bodies and organs. Specifically, the present invention can be used to model genetic disorders and identify drug molecules for the treatment of disorders such as myotonic dystrophy and van Waardenburg syndrome.

The principles and operation of the stem cells which carry disease-causing mutations of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Genetic disorders result from chromosomal aberrations and/or DNA abnormalities which are transmitted in a recessive (e.g., cystic fibrosis and Canavan), dominant (e.g., Myotonic Dystrophy) or imprinting (e.g., Prader-Willi or Angelman syndromes) mode of inheritance.

Continuous efforts in the field of genetics, and especially, in human genetics, resulted in various diagnostic tools for many genetic disorders. Thus, chromosomal and DNA abnormalities can be diagnosed in affected individuals, un-affected carriers (e.g., of a recessive disorder) and in embryos, using chorionic villi and amniotic fluid samples, or even prior to the implantation of an in vitro fertilized embryo. However, for many genetic disorders, the processes leading to the overall disorder's phenotype are still unknown.

Prior attempts to reveal the molecular and physiological basis of genetic disorders include the generation of several disease-models, such as cell cultures and animal models (Vogan K J, et al., 1993, Genomics. 17: 364-9; Asher et al, 1996; Berul C I, et al., 2000, J. Interv. Card. Electrophysiol. 4: 351-8; Amack J D, et al., 1999, Hum. Mol. Genet. 8: 1975-84; van Doorninck J H, et al., 1995, EMBO J. 14: 4403-11; Dickinson P et al., 2002, Mol. Genet. 11: 243-51; D'Hooge R, et al., 2001, Brain Res. 907: 35-43). However, although such disease-models present biochemical models of the disorder, they often do not reproduce the disorder's clinical symptoms (Elsea S H, Lucas R E., 2002, ILAR J. 43: 66-79). Thus, in most cases, the presently available disease-models are not suitable for drug development.

While reducing the present invention to practice the present inventors have uncovered that embryos carrying naturally occurring disease-causing mutations can be used to generate ES cell lines and that such ES cell lines can be further used in developing cure for genetic disorders.

As is shown in Example 1 of the Examples section which follows the present inventors have successfully generated ES cell lines carrying disease-causing mutations for the van Waardenburg syndrome, Myotonic Dystrophy, metachromatic leukodystrophy and cystic fibrosis.

Thus, according to one aspect of the present invention there is provided an isolated stem cell or stem cell line carrying a disease-causing mutation in a genomic polynucleotide sequence thereof.

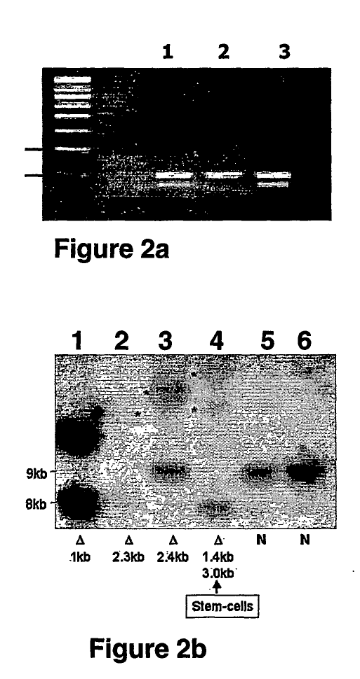

For example, as is shown in FIGS. 2a-b and in Example 1 of the Examples section which follows, the I-5 and I-7 ES cell line carry the deletion of 28 bp in the Pax3 gene and abnormal (i.e., more than 50) repeats of the CTG trinucleotide of the DMPK, gene causing van Waardenburg syndrome and Myotonic Dystrophy, respectively.

As used herein, the phrase “stem cell” refers to a cell capable of differentiating into other cell types having a particular, specialized function (i.e., “fully differentiated” cells) or to cells capable of being maintained in an undifferentiated state, hereinafter “pluripotent stem cells” or partially differentiated state, herein “multipotent stem cells”.

The stem cell of the present invention can be an hematopoietic stem cell obtained from bone marrow tissue of an individual at any age or from cord blood of a newborn individual, an adult tissue stem cell derived from an adult tissue (e.g., adipose tissue, skin, kidney, liver, prostate, pancreas, intestine, and bone marrow), or an embryonic stem (ES) cell obtained from the embryonic tissue formed after gestation (e.g., blastocyst), or embryonic germ (EG) cells.

As is mentioned hereinabove, the stem cell of the present invention is preferably of embryonic origin [i.e., embryonic stem (ES) or embryonic germ (EG) cells]. ES and EG cells can differentiate into cells of all embryonic germ layers, i.e., the endoderm, ectoderm and mesoderm and developing into all types of cells, tissues, organs and/or body parts, including a whole organism.

ES or EG cell carrying a disease-causing mutation can be prepared using methods known in the arts.

ES cells can be isolated from blastocysts which are obtained from in vivo preimplantation embryos or from in vitro fertilized (IVF) embryos. Alternatively, a single cell embryo can be expanded to the blastocyst stage. For the isolation of ES cells the zona pellucida is removed from the blastocyst, or digested using Tyrode's acidic solution (Sigma, St Louis, Mo., USA) and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. For the derivation of human ES cells, following 9 to 15 days in culture, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 1-2 weeks. For further details on methods of preparation ES cells see Example 1 of the Examples section which follows and Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989] and Gardner et al., [Fertil. Steril. 69: 84, 1998].

EG cells can be prepared from the primordial germ cells. For human EG cells, the primordial germ cells are obtained from human fetuses of about 8-11 weeks of gestation using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

ES cells can be obtained from a variety of sources including human (Amit M and Itskovitz-Eldor J., 2002, J, Anat, 200: 225), mouse (Mills A A and Bradley A, 2001, Trends Genet. 17: 331-9), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species such as Rhesus monkey and marmoset (Thomson et al., 1995, Proc Natl Acad Sci U S A. 92: 7844-8; Thomson et al., 1996, Biol Reprod. 55: 254-9). The ES cells are obtained from any source which can carry the genetic disorder, such a source can be an animal model of the disease or a human embryo which naturally carries the genetic disorder. For example, ES cells can be obtained from domestic pigs embryos carrying the G590R mutation in the alpha1 (X) chain of type X collagen which is associated with dwarfism (Nielsen V H et al., Mamm Genome. 2000; 11: 1087-92), mice embryos carrying the 1-bp insertion (267-268 insC, codon 90 in the Cln8 gene) which is associated with motor neuron degeneration (Ranta S et al., Nat Genet. 1999; 23: 233-6), feline model of mucopolysaccharidosis type VI (Nuttall J D et al., Calcif Tissue Int. 1999; 65: 47-52) and mice embryos carrying the no b-wave (nob) X-linked recessive mutation, which is a model of congenital stationary night blindness (Pardue M T et al., Invest Ophthalmol Vis Sci. 1998; 39: 2443-9). The presence of a disease-causing mutation in such ES cells can be identified using molecular and cytogenetic methods known in the art which are listed hereinbelow.

Although less preferred, the stem cell of the present can be an hematopoietic stem cell provided from bone marrow cells, mobilized peripheral blood cells or cord blood cells. For example, hematopoietic stem cell can be obtained from cord blood of fetuses carrying mutations in the IL2RG, ARTEMIS, RAG1, RAG2, ADA, CD45, JAK3, or IL7R genes which cause severe combined immunodeficiency (SCID, Kalman L et al., Genet Med. 2004; 6: 16-26), from fetuses or adults carrying mutations in the Wiskott-Aldrich syndrome (WAS) gene which are associated with congenital thrombocytopenia (Luthi J N et al., Exp Hematol. 2003; 31: 150-8) and from fetuses or adults carrying the 5881G>T mutation in the erythropoietin receptor (EPOR) gene which is associated with primary familial erythrocytosis (familial polycythentia, Arcasoy M O et al., Blood. 2002; 99: 3066-9). Bone marrow cells can be obtained from the donor by standard bone marrow aspiration techniques know in the art, for example by aspiration of marrow from the iliac crest. Peripheral blood stem cells are obtained after stimulation of the donor with a single or several doses of a suitable cytokine, such as granulocyte colony-stimulating factor (G-CSF), granulocyte/macrophage colony-stimulating factor (GM-CSF) and interleukin-3 (IL-3). In order to harvest desirable amounts of stem cells from the peripheral blood cells, leukapheresis is performed by conventional techniques (Caspar, C. B. et al., 1993. Blood. 81: 2866-71) and the final product is tested for mononuclear cells. Cord blood cells are obtained from newborn individuals. Nucleated cells are separated from erythrocytes using methods known in the arts such as a bag system and separation by agglutination (see International Publication No. WO 96/17514). CD43 expressing hematopoietic stem cells are enriched using combinations of density centrifugation, immuno-magnetic bead purification, affinity chromatography, and fluorescent active cell sorting (FACS). CD34+ enriched stem cells are then cultured in the presence of growth factors such as IL-3 and stem cell factor.

Alternatively and presently less preferred, the stem cell of the present invention can be an adult tissue stem cell which can be isolated using methods known in the arts [Alison, M. R., J. Pathol. 2003 200(5): 547-50; Cai, J. et al., Blood Cells Mol Dis. 2003 31(1): 18-27; and Collins, A. T. et al., J Cell Sci. 2001; 114(Pt 21): 3865-72]. For example, adult tissue stem cells can be obtained from individuals having somatic mutations in the pluripotential stem cell which causes myelodysplastic syndromes (Narayan S et al,. Pediatr Dermatol. 2001; 18: 210-2).

The phrase “stem cell line” refers to a population of stem cells which are derived from stem cells and have been maintained in culture for an extended period of time, i.e., for a time period which allows stem cell expansion for at least 10⁶ cells.

The phrase “disease-causing mutation” refers to any chromosomal and/or DNA abnormality which is capable of causing a disease, disorder or condition and/or an alteration in a phenotype which is associated with the disease, disorder or condition.

The phrase “genomic polynucleotide sequence” refers to any DNA or RNA polynucleotide sequence which is derived from the stem cell or stem cell line of the present invention.

Examples for disease-causing mutations generated by chromosomal abnormalities include, but are not limited to trisomies (e.g., Down Syndrome), monosomies (e.g., Turner's syndrome), deletions (e.g., DiGeorge syndrome), duplications (e.g., Silver-Russell syndrome), translocations (e.g., Beckwith-Wiedemann) and inversions (e.g., Hypogonadotropic hypogonadism).

Such chromosomal abnormalities can be identified using methods known in the arts, including chromosomal banding (e.g., G-banding, R-banding), fluorescent in situ hybridization (FISH), primed in situ labeling (PRINS), multicolor-banding (MCB) and/or quantitative FISH (Q-FISH).

Examples for disease-causing mutations generated by DNA abnormalities (e.g., single nucleotide substitution, deletion, insertion, or repeat expansion) include, but are not limited to, a missense mutation (i.e., a mutation which changes an amino acid residue in the protein with another amino acid residue), a nonsense mutation (i.e., a mutation which introduces a stop codon in a protein), a frameshift mutation (i.e., a mutation, usually, deletion or insertion of nucleic acids which changes the reading frame of the protein, and may result in an early termination or in a longer amino acid sequence), a readthrough mutation (i.e., a mutation which results in an elongated protein due to a change in a coding frame or a modified stop codon), a promoter mutation (i.e., a mutation in a promoter sequence, usually 5′ to the transcription start site of a gene, which result in up-regulation or down-regulation of a specific gene product), a regulatory mutation (i.e., a mutation in a region upstream or downstream, or within a gene, which affects the expression of the gene product), a deletion (i.e., a mutation which deletes coding or non-coding nucleic acids in a gene sequence), an insertion (i.e., a mutation which inserts coding or non-coding nucleic acids into a gene sequence), an inversion (i.e., a mutation which results in an inverted coding or non-coding sequence), a splice mutation (i.e., a mutation which results in abnormal splicing or poor splicing) and a duplication (i.e., a mutation which results in a duplicated coding or non-coding sequence).

Following is a non-limiting list of methods which can be used to identify nucleic acid substitutions in the stem cell or stem cell line of the present invention which result in disease-causing mutations.

Direct sequencing of a PCR product: This method is based on the amplification of a genomic sequence using specific PCR primers in a PCR reaction following by a sequencing reaction utilizing the sequence of one of the PCR primers as a sequencing primer. Sequencing reaction can be performed using, for example, the Applied Biosystems (Foster City, Calif.) ABI PRISM® BigDye™ Primer or BigDye™ Terminator Cycle Sequencing Kits.

Restriction fragment length polymorphism (RFLP): This method uses a change in a single nucleotide which modifies a recognition site for a restriction enzyme resulting in the creation or destruction of an RFLP.

For example, RFLP can be used to detect the cystic fibrosis—causing mutation, ΔF508 [deletion of a CTT at nucleotide 1653-5, GenBank Accession No. M28668, SEQ ID NO:24; Kerem B, et al., Science. 1989, 245: 1073-80] in a genomic DNA derived from the stem cell or stem cell line of the present invention. Briefly, genomic DNA is amplified using the forward [5′-GCACCATTAAAGAAAATATGAT (SEQ ID NO:25)] and the reverse [5′-CTCTTCTAGTTGGCATGCT (SEQ ID NO:26)] PCR primers, and the resultant 86 or 83 bp PCR products of the wild-type or AF508 allele, respectively are subjected to digestion using the DpnI restriction enzyme which is capable of differentially digesting the wild-type PCR product (resulting in a 67 and 19 bp fragments) but not the CTT-deleted allele (resulting in a 83 bp fragment).

Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

Allele specific oligonucleotide (ASO): In this method, an allele-specific oligonucleotide (ASO) is designed to hybridize in proximity to the polymorphic nucleotide, such that a primer extension or ligation event can be used as the indicator of a match or a mis-match. Hybridization with radioactively labeled allelic specific oligonucleotides (ASO) also has been applied to the detection of specific SNPs (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles.

It will be appreciated that ASO can be applied on a PCR product generated from genomic DNA. For example, to detect the A455E mutation (C1496→A in SEQ ID NO:24) which causes cystic fibrosis, genomic DNA (of the stem cell or stem cell line of the present invention) is amplified using the 5′-TAATGGATCATGGGCCATGT (SEQ ID NO:27) and the 5′-ACAGTGTTGAATGTGGTGCA (SEQ ID NO:28) PCR primers, and the resultant PCR product is subjected to an ASO hybridization using the following oligonucleotide probe: 5′-GTTGTTGGAGGTTGCT (SEQ ID NO:29) which is capable of hybridizing to the thymidine nucleotide at position 1496 of SEQ ID NO:1. As a control for the hybridization, the 5′-GTTGTTGGCGGTTGCT (SEQ ID NO:30) oligonucleotide probe is applied to detect the presence of the wild-type allele essentially as described in Kerem B, et al., 1990, Proc. Natl. Acad. Sci. USA, 87:8447-8451).

Allele-specific PCR—In this method the presence of a single nucleic acid substitution is detected using differential extension of a mutant and/or wild-type—specific primer on one hand, and a common primer on the other hand. For example, the detection of the cystic fibrosis Q493X mutation (C1609→T in SEQ ID NO:24) is performed by amplifying genomic DNA (derived from the stem cell or stem cell line of the present invention) using the following three primers: the common primer (i.e., will amplify in any case): 5′-GCAGAGTACCTGAAACAGGA (SEQ ID NO:31); the wild-type primer (i.e., will amplify only the cytosine-containing wild-type allele): 5′-GGCATAATCCAGGAAAACTG (SEQ ID NO:32); and the mutant primer (i.e., will amplify only the thymidine-containing mutant allele): 5′-GGCATAATCCAGGAAAACTA (SEQ ID NO:33), essentially as described in Kerem, 1990 (Supra).

Methylation-specific PCR (MSPCR)—This method is used to detect specific changes in DNA methylation which are associated with imprinting disorders such Angelman or Prader-Willi syndromes. Briefly, the DNA is treated with sodium bisulfite which converts the unmethylated, but not the methylated, cytosine residues to uracil. Following sodium bisulfite treatment the DNA is subjected to a PCR reaction using primers which can anneal to either the uracil nucleotide-containing allele or the cytosine nucleotide-containing allele as described in Buller A., et al., 2000, Mol. Diagn.5: 239-43.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of a single nucleotide substitution (i.e., the disease-causing mutation of the present invention) in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of SNPs.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walling, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Pyrosequencing™ analysis (Pyrosequencing, Inc. Westborough, Mass., USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a pyrogram™. Each light signal is proportional to the number of nucleotides incorporated.

Acycloprime™ analysis (Perkin Elmer, Boston, Mass., USA): This technique is based on fluorescent polarization (FP) detection. Following PCR amplification of the sequence containing the SNP of interest, excess primer and dNTPs are removed through incubation with shrimp alkaline phosphatase (SAP) and exonuclease I. Once the enzymes are heat inactivated, the Acycloprime-FP process uses a thermostable polymerase to add one of two fluorescent terminators to a primer that ends immediately upstream of the site of the single nucleotide substitution. The terminator(s) added are identified by their increased FP and represent the allele(s) present in the original DNA sample. The Acycloprime process uses AcycloPol™, a novel mutant thermostable polymerase from the Archeon family, and a pair of AcycloTerminators™ labeled with R110 and TAMRA, representing the possible alleles for the SNP of interest. AcycloTerminator™ non-nucleotide analogs are biologically active with a variety of DNA polymerases. Similarly to 2′,3′-dideoxynucleotide-5′-triphosphates, the acyclic analogs function as chain terminators. The analog is incorporated by the DNA polymerase in a base-specific manner onto the 3′-end of the DNA chain, and since there is no 3′-hydroxyl, is unable to function in further chain elongation. It has been found that AcycloPol has a higher affinity and specificity for derivatized AcycloTerminators than various Taq mutant have for derivatized 2′,3′-dideoxynucleotide terminators.

Reverse dot blot: This technique uses labeled sequence specific oligonucleotide probes and unlabeled nucleic acid samples. Activated primary amine-conjugated oligonucleotides are covalently attached to carboxylated nylon membranes. After hybridization and washing, the labeled probe, or a labeled fragment of the probe, can be released using oligomer restriction, i.e., the digestion of the duplex hybrid with a restriction enzyme. Circular spots or lines are visualized colorimetrically after hybridization through the use of streptavidin horseradish peroxidase incubation followed by development using tetramethylbenzidine and hydrogen peroxide, or via chemiluminescence after incubation with avidin alkaline phosphatase conjugate and a luminous substrate susceptible to enzyme activation, such as CSPD, followed by exposure to x-ray film.

It will be appreciated that the disease-causing mutation of the present invention can be identified using various advanced single nucleotide polymorphism (SNP) genotyping techniques, such as dynamic allele-specific hybridization (DASH, Howell, W. M. et al., 1999. Dynamic allele-specific hybridization (DASH). Nat. Biotechnol. 17: 87-8), microplate array diagonal gel electrophoresis [MADGE, Day, I. N. et al., 1995. High-throughput genotyping using horizontal polyacrylamide gels with wells arranged for microplate array diagonal gel electrophoresis (MADGE). Biotechniques. 19: 830-5], the TaqMan system (Holland, P. M. et al., 1991. Detection of specific polymerase chain reaction product by utilizing the 5′→3′ exonuclease activity of Thermus aquaticus DNA polymerase. Proc Natl Acad Sci U S A. 88: 7276-80), as well as various DNA “chip” technologies such as the GeneChip microarrays (e.g., Affymetrix SNP chips) which are disclosed in U.S. patent application Ser. No. 6,300,063 to Lipshutz, et al. 2001, which is fully incorporated herein by reference, Genetic Bit Analysis (GBA™) which is described by Goelet, P. et al. (PCT Appl. No. 92/15712), peptide nucleic acid (PNA, Ren B, et al., 2004. Nucleic Acids Res. 32: e42) and locked nucleic acids (LNA, Latorra D, et al., 2003. Hum. Mutat. 22: 79-85) probes, Molecular Beacons (Abravaya K, et al., 2003. Clin Chem Lab Med. 41: 468-74), intercalating dye [Germer, S. and Higuchi, R. Single-tube genotyping without oligonucleotide probes. Genome Res. 9:72-78 (1999)], FRET primers (Solinas A et al., 2001. Nucleic Acids Res. 29: E96), AlphaScreen (Beaudet L, et al., Genome Res. 2001, 11(4): 600-8), SNPstream (Bell Pa., et al., 2002. Biotechniques. Suppl.: 70-2, 74, 76-7), Multiplex minisequencing (Curcio M, et al., 2002. Electrophoresis. 23: 1467-72), SnaPshot (Turner D, et al., 2002. Hum Immunol. 63: 508-13), MassEXTEND (Cashman J R, et al., 2001. Drug Metab Dispos. 29: 1629-37), GOOD assay (Sauer S, and Gut I G. 2003. Rapid Commun. Mass. Spectrom. 17: 1265-72), Microarray minisequencing (Liljedahl U, et al., 2003. Pharmacogenetics. 13: 7-17), arrayed primer extension (APEX) (Tonisson N, et al., 2000. Clin. Chem. Lab. Med. 38: 165-70), Microarray primer extension (O'Meara D, et al., 2002. Nucleic Acids Res. 30: e75), Tag arrays (Fan J B, et al., 2000. Genome Res. 10: 853-60), Template-directed incorporation (TDI) (Akula N, et al., 2002. Biotechniques. 32: 1072-8), fluorescence polarization (Hsu T M, et al., 2001. Biotechniques. 31: 560, 562, 564-8), Colorimetric oligonucleotide ligation assay (OLA, Nickerson D A, et al., 1990. Proc. Natl. Acad. Sci. USA. 87: 8923-7), Sequence-coded OLA (Gasparini P, et al., 1999. J. Med. Screen. 6: 67-9), Microarray ligation, Ligase chain reaction, Padlock probes, Rolling circle amplification, Invader assay (reviewed in Shi M M. 2001. Enabling large-scale pharmacogenetic studies by high-throughput mutation detection and genotyping technologies. Clin Chem. 47: 164-72), coded microspheres (Rao K V et al., 2003. Nucleic Acids Res. 31: e66) and MassArray (Leushner J, Chiu N H, 2000. Mol Diagn. 5: 341-80).

It will be appreciated that nucleic acid substitutions can be also identified in mRNA molecules derived from the stem cell or stem cell line of the present invention. Such mRNA molecules are first subjected to an RT-PCR reaction following which they are either directly sequenced or be subjected to any of the SNP detection methods described hereinabove.

The disease-causing mutations of the present invention can be present in the stem cell or stem cell line of the present invention in a heterozygous (i.e., the presence of only one disease-causing mutation), homozygous (i.e., the presence of two identical disease-causing mutations), or double heterozygous (i.e., the presence of two different disease-causing mutations) form. It will be appreciated that the mode of inheritance of the disease-causing mutation (i.e., dominant, recessive, co-dominant and/or imprinting) can affect the outcome of the mutation, i.e., the presence or absence of the alteration of the phenotype of the stem cell or stem cell line of the present invention.

Thus, while in the case of a dominant disorder (e.g., Myotonic dystrophy) stem cell or stem cell line which are heterozygote for a disease-causing mutation exhibit the alteration of the phenotype, in the case of a recessive disorder, only stem cells or stem cell line which are homozygous or double-heterozygous to disease-causing mutations exhibit the alteration of the phenotype.

As is shown in Example 1 of the Examples section which follows, the present inventors have isolated the I-5 ES cell line which carries the PAX3-del28 (510del28 in SEQ ID NO:34) in a heterozygous form and which is associated with van Waardenburg syndrome; the I-7 ES cell line which carries more than 50 repeats of the CTG trinucleotide as set forth in SEQ ID NO:22 in a heterozygous form and which is associated with Myotonic dystrophy; the I-8. and I-9 which carry the 1505C→T (P377L) mutation as set forth in SEQ ID NO:21 in a heterozygout form and which is associated with metachromatic leukodystrophy and the J-3 ES cell line which carries the W1282X mutation as set forth in SEQ ID NO:24 in a heterozygous form and which is associated with cystic fibrosis.

As used herein, the phrase “alteration of the phenotype” refers to changes in the shape and function of the cells including, but not limited to changes in receptor binding, cell secretion, intracellular reactions which lead to upregulation or downregulation of certain genes, changes in the size and shape of the cells and/or the cellular compartments (e.g., nucleus, cytoplasm, nucleolus), changes in proliferation and/or differentiation processes of the cells, and the like. More specifically, the alteration of the phenotype of the present invention can be lysosomal accumulation of sulfatides in Schwann cells, periaxonal Schwann cells, macrophages, and spiral and vestibular ganglion cell perikarya due to mutations causing metachromatic leukodystrophy (Coenen R, et al., cta Neuropathol (Berl). 2001; 101: 491-8); defects in cAMP-activated whole-cell currents and Cl— transport in cell lines carrying cystic fibrosis mutations (Zamecnik P C et al., Proc Natl Acad Sci U S A. 2004; 101: 8150-5); and defects in migration and differentiation in muscle and neuronal cells carrying Myotonic dystrophy mutations (Yanowitz J L et al., Dev Biol. August 2004 15;272(2):389-402).

It will be appreciated that such alterations in the phenotype can be detected using histological stains (May-Grünwald-Giemsa stain, Giemsa stain, Papanicolau stain, Hematoxyline stain and/or DAPI stain), flow cytometry analysis of membrane bound markers using, e.g., a fluorescence-activated cell sorting (FACS), biochemical assays (e.g., using enzymatic assays), immunological assays (e.g., using specific antibodies), and/or RNA assays (e.g., using RT-PCR, Northern blot, RNA in situ hybridization and in situ RT-PCR), cell proliferation assays [e.g., using a MTT-based cell proliferation assay (Hayon, T. et al., 2003. Leuk Lymphoma. 44: 1957-62)], cell differentiation assays (Kohler, T., et al., 2000. Stem Cells.18: 139-47), apoptosis assays [e.g., using the Ethidium homodimer-1 (Molecular Probes, Inc., Eugene, Oreg., USA), the Tunnel assay (Roche, Basel, Switzerland), the live/dead viability/cytotoxicity two-color fluorescence assay (L-3224, Molecular Probes)], flow cytometry analysis [Lodish, H. et al., “Molecular Cell Biology”, W. H. Freeman (Ed.), 2000], and the like.

In order to generate the isolated stem cell or stem cell line of the present invention, a single stem cell which carry a disease-causing mutation is isolated as described hereinabove from a human embryo carrying a disease-causing mutation (e.g., van Waardenburg syndrome, Myotonic dystrophy) and preferably cultured. Such a human embryo can be an embryo (at the blastocyst stage) which was subjected to pre-implantation genetic diagnosis (POD) and was found to carry disease-causing mutations. Methods of culturing ES cells are known in the arts. Briefly, stem cells are plated on a matrix (e.g., Matrigel®™) or feeder cell layers (e.g., MEFs, foreskin feeder cells) in a cell density which promotes cell survival and proliferation but limits differentiation. Typically, a plating density of between about 15,000 cells/cm² and about 200,000 cells/cm² is used.

It will be appreciated that although single-cell suspensions of stem cells are usually seeded, small clusters may also be used. To this end, enzymatic digestion utilized for cluster disruption (see Example 1 of the Examples section which follows) is terminated before stem cells become completely dispersed and the cells are triturated with a pipette such that clumps (i.e., 10-200 cells) are formed. However, measures are taken to avoid large clusters which cause cell differentiation.

According to preferred embodiments of the present invention, the culture medium includes cytokines and growth factors needed for cell proliferation [e.g., basic fibroblast growth factor (bFGF) and leukemia inhibitor factor (LIF)], and factors such as transforming growth factor β₁ (TGFβ₁) which inhibit stem cell differentiation.

Such a culture medium can be a synthetic tissue culture medium such as Ko-DMEM (Gibco-Invitrogen Corporation products, Grand Island, N.Y., USA) supplemented with serum, serum replacement and/or growth factors.

Serum can be of any source including fetal bovine serum (FBS), defined FBS (HyClone, Utah, USA), goat serum, human serum and/or serum replacement™ (Gibco-Invitrogen Corporation, Grand Island, N.Y. USA).

Culture medium, serum, and serum replacement can be obtained from any commercial supplier of tissue culture products, examples include Gibco-Invitrogen Corporation (Grand Island, N.Y. USA), Sigma (St. Louis Mo., USA), HyClone (Utah, USA) and the ATCC (Manassas, Va. USA).

The serum or serum replacement used by the present invention are provided at a concentration range of 1% to 40%, more preferably, 5% to 35%, most preferably, 10% to 30%.

Growth factors of the present invention can be used at any combination and can be provided to the stem cells at any concentration suitable for ES cell proliferation, while at the same time inhibit ES cell differentiation.

As shown in Example 1 of the Examples section which follows, the ES cells of the present invention which carry the disease-causing mutations were cultured on MEFs in the presence of culture medium (80% KO-DMEM) supplemented with 20% defined FBS, 1 mM L-glutamine, 0.1 mM β-mercaptoethanol, 1% non-essential amino acid stocks and were maintained in an undifferentiated state for at least 40 passages.

Alternatively, culturing the hES cells of the present invention can be effected using a conditioned medium instead of serum or serum replacement supplemented medium.

Conditioned medium is the growth medium of a monolayer cell culture (i.e., feeder cells) present following a certain culturing period. The conditioned medium includes growth factors and cytokines secreted by the monolayer cells in the culture.

Conditioned medium can be collected from a variety of cells forming monolayers in culture. Examples include MEF conditioned medium, foreskin conditioned medium, human embryonic fibroblasts conditioned medium, human fallopian epithelial cells conditioned medium, and the like.

Particularly suitable conditioned medium are those derived from human cells, such as foreskin-conditioned medium which is produced by culturing human foreskin cells in a growth medium under conditions suitable for producing the conditioned medium.

Such a growth medium can be any medium suitable for culturing feeder cells. The growth medium can be supplemented with nutritional factors, such as amino acids, (e.g., L-glutamine), anti-oxidants (e.g., beta-mercaptoethanol) and growth factors, which benefit stem cell growth in an undifferentiated state. Serum and serum replacements are added at effective concentration ranges as described elsewhere (U.S. patent application Ser. No. 10/368,045).

Feeder cells are cultured in the growth medium for sufficient time to allow adequate accumulation of secreted factors to support stem cell proliferation in an undifferentiated state. Typically, the medium is conditioned by culturing for 4-24 hours at 37° C. However, the culturing period can be scaled by assessing the effect of the conditioned medium on stem cell growth and differentiation.

Selection of culture apparatus for conditioning the medium is based on the scale and purpose of the conditioned medium. Large-scale production preferably involves the use of dedicated devices. Continuous cell culture systems are reviewed in Furey (2000) Genetic Eng. News 20:10.

Following accumulation of adequate factors in the medium, growth medium (i.e., conditioned medium) is separated from the feeder cells and collected. It will be appreciated that the feeder cells can be used repeatedly to condition further batches of medium over additional culture periods, provided that the cells retain their ability to condition the medium.

Preferably, the conditioned medium is sterilized (e.g., filtration using a 20 μM filter) prior to use. The conditioned medium of the present invention may be applied directly on stem cells or extracted to concentrate the effective factor such as by salt filtration. For future use, conditioned medium is preferably stored frozen at −80° C.

During the culturing step the stem cells are monitored for their differentiation state. Typically, undifferentiated stem cells have high nuclear/cytoplasmic ratios, prominent nucleoli and compact colony formation with poorly discernable cell junctions.

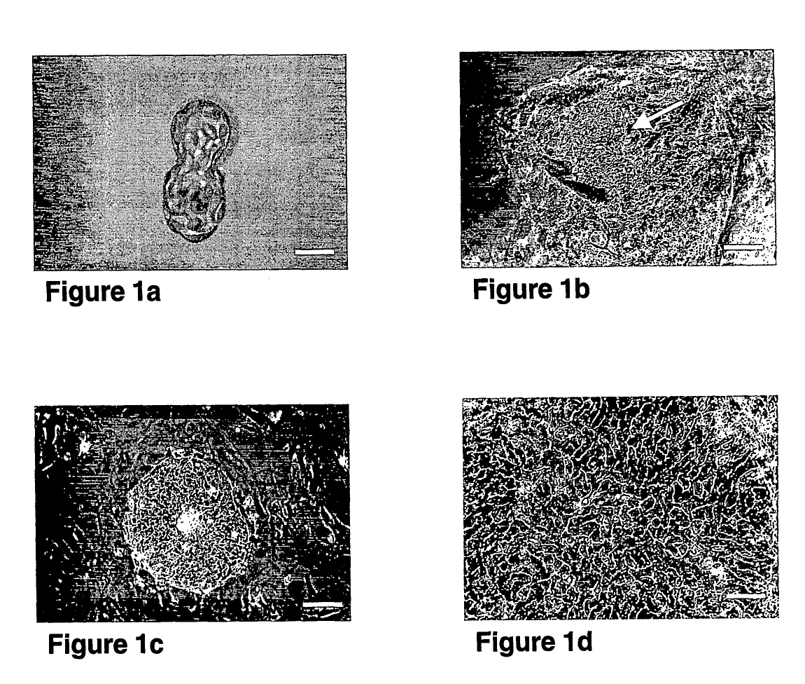

As is shown in Example 1 of the Examples section which follows and in FIGS. 1c-d, the present inventors have illustrated that the ES cells of the present invention which carry the disease-causing mutation display characteristic morphology of undifferentiated ESCs, i.e., round colonies, clear borders, spaces between cells, high cytoplasm to nucleus ratio and existence of two or four nucleoli.

Cell differentiation can be determined upon examination of cell or tissue-specific markers which are known to be indicative of differentiation. Such tissue/cell specific markers can be detected using immunological techniques well known in the art [Thomson J A et al., (1998). Science 282: 1145-7]. Examples include, but are not limited to, flow cytometry for membrane-bound markers, immunohistochemistry for extracellular and intracellular markers and enzymatic immunoassay, for secreted molecular markers. Thus, primate ES cells may express the stage-specific embryonic antigen (SSEA) 4, the tumor-rejecting antigen (TRA)-1-60 and TRA-1-81.

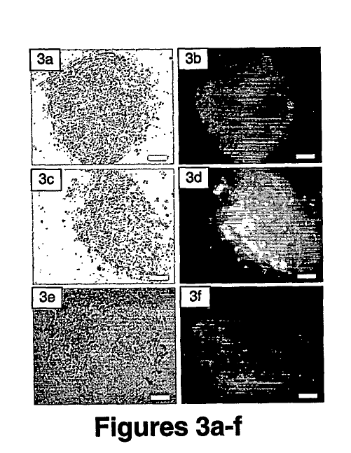

As is shown in FIGS. 3a-f in Example 1 of the Examples section which follows, ES cells carrying the Van Waardenburg disease-causing mutation of the present invention expressed the SSEA4, TRA-1-60 and TRA-1-81 cell surface markers typical for undifferentiated cells.

Determination of ES cell differentiation can also be effected via measurements of alkaline phosphatase activity. Undifferentiated human ES cells have alkaline phosphatase activity which can be detected by fixing the cells with 4% paraformaldehyde and developing with the Vector Red substrate kit according to manufacturer's instructions (Vector Laboratories, Burlingame, Calif., USA).

As is shown in Example 1 of the Examples section which follows, the I-5 and I-7 stem cells which carry the WS1 and DM1 mutations, respectively, remained in an undifferentiated proliferation state for at least 41 passages.

In addition to monitoring a differentiation state, stem cells are often also being monitored for karyotype, in order to verify cytological euploidity, wherein all chromosomes are present and not detectably altered during culturing. Cultured stem cells can be karyotyped using a standard Giemsa staining and compared to published karyotypes of the corresponding species.

The stem cells of the present invention which carry disease-causing mutations of the WS1, DM1, CF and MLD genetic disorders retain a normal karyotype i.e., 46, XX or 46, XY following at least 30 passages (see Example 1 of the Examples section).

It will be appreciated that the stem cell or stem cell line of the present invention which carry the disease-causing mutation are likely to pass the disease-causing mutation to any differentiated cell, tissue or organ which is derived thereof.

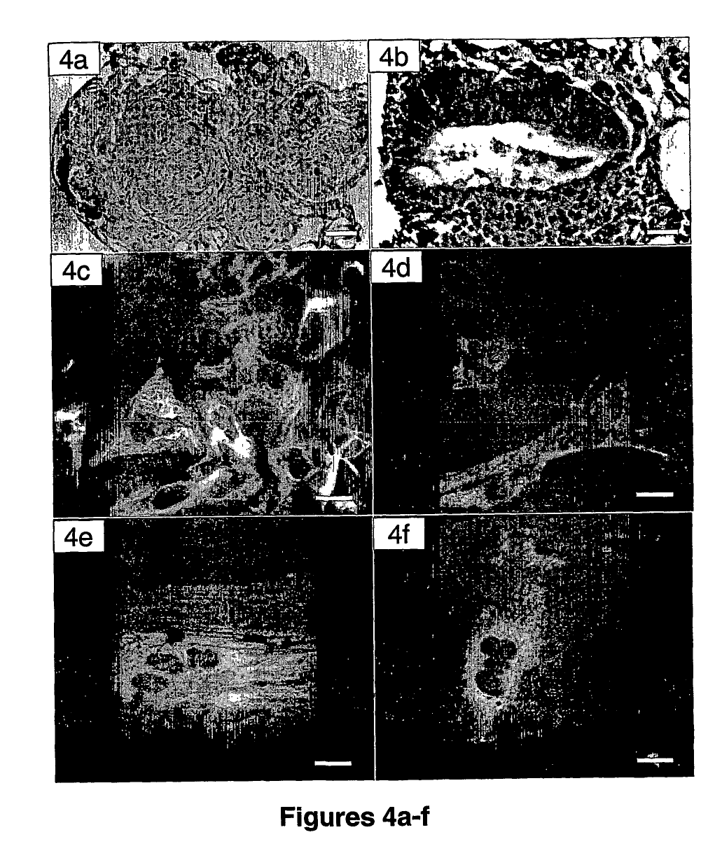

As is shown in Example 2 of the Examples section which follows and in FIGS. 4c-f, 5 and 6a-d, the I-5 and I-7 ES cells were capable of differentiating in vitro (embryoid bodies) and in vivo (teratomas) to all three embryonic germ layers, namely, ectoderm, mesoderm and endoderm. Such a pluripotent capacity was retained even following 40 passages.

Thus, according to another aspect of the present invention there is provided an isolated embryoid body comprising a plurality of cells at least some of which carry a disease-causing mutation in a genomic polynucleotide sequence thereof.

As used herein, the phrase “embryoid body” (EB) refers to morphological structures comprised of a population of ES and/or EG cells which have undergone differentiation. EBs formation initiates following the removal of differentiation blocking factors from ES cell cultures. In the first step of EBs formation, ES cells proliferate into small masses of cells which then proceed with differentiation. In the first phase of differentiation, following 1-4 days in culture for human ES cells, a layer of endodermal cells is formed on the outer layer of the small mass, resulting in “simple EBs”. In the second phase, following 3-20 days post-differentiation, “complex EBs” are formed. Complex EBs are characterized by extensive differentiation of ectodermal and mesodermal cells and derivative tissues.

The phrase “at least some” as used herein refers to a situation of genetic mosaicism in which the embryoid body was formed from a group of stem cells part of which was carrying the disease-causing mutation of the present invention. According to preferred embodiments “at least some” refers to at least 1%, more preferably, at least 2%, more preferably, at least 3%, at least 4%, 5%, 6%, 7%, 8%, 9%, 10,%, 11%, more preferably, between 12%-98%, more preferably, between 20%-80%, more preferably, between 30-60%, most preferably, at least 50% of the cells carry the disease-causing mutation of the present invention.

As is mentioned above, EBs are formed following the removal of ES cells from feeder layer-, or matrix-based cultures into suspension cultures. ES cells removal can be effected using type IV Collagenase treatment for a limited time. Following dissociation from the culturing surface, the cells are transferred to tissue culture plates containing a culture medium supplemented with serum and amino acids.

It will be appreciated that EBs can be collected at any time during culturing and examined using an inverted light microscope. Thus, EBs can be assessed for their size and shape at any point in the culturing period. Examples of various EBs structures are shown in FIGS. 4a-b.

During the culturing step, EBs can be monitored for their viability using methods known in the arts, including, but not limited to, DNA (Brunk, C. F. et al., Analytical Biochemistry 1979, 92: 497-500) and protein (e.g., using the BCA Protein Assay kit, Pierce, Technology Corporation, New York, N.Y., USA) contents, medium metabolite indices, e.g., glucose consumption, lactic acid production, LDH (Cook J. A., and Mitchell J. B. Analytical Biochemistry 1989, 179: 1-7) and medium acidity, as well as by using the XTT method of detecting viable cells [Roehm, N. et al., J. Immunol. Meth. 142, 257-265 (1991); Scudierd, D. et al., Cancer Res. 48, 4827-4833 (1988); Weislow, O. et al., J. Natl. Cancer Inst. 81, 577-586 (1989)].

In addition, the viability of the EBs of the present invention can be also assessed using various staining methods, including but not limited to the fluorescent Ethidium homodimer-1 dye (excitation, 495 nm; emission, 635 nm) which is detectable in cells with compromised membranes, i.e., dead cells; the Tunnel assay which labels DNA breaks characteristics of cells going through apoptosis; and the live/dead viability/cytotoxicity two-color fluorescence assay, available from Molecular Probes (L-3224, Molecular Probes, Inc., Eugene, Oreg., USA).

The differentiation level of the EB cells can be monitored by following the loss of expression of Oct-4, and the increased expression level of other markers such as α-fetoprotein, NF-68 kDa, α-cardiac and albumin. Methods useful for monitoring the expression level of specific genes are well known in the art and include RT-PCR, RNA in situ hybridization, Western blot analysis and immunohistochemistry.

As is shown in FIGS. 4c-f and 5, the EBs of the present invention which carry the WS1 or DM1 disease-causing mutations expressed neurofilament 68 KD and nestin which represent the ectoderm layer, α-cardiac actin and troponin which represent the mesoderm layer and albumin and insulin which represent the endoderm layer. In addition, the diminished Oct-4 expression in 5-day-old EBs demonstrate the decrease in undifferentiated ES cells along with EB formation.

As is mentioned above, EBs are cultured in suspension cultures in the presence of a culture medium suitable for EB differentiation. Preferably, such a culture medium also includes serum or serum replacement, which are provided in a concentration of at least 10% or 15%, respectively.

The EBs of the present invention can be at any age. Preferably, the EBs of the present invention are between 1-120 day-old, more preferably between 1-30 day-old, 1-10 day-old, more preferably, between 2-10 day-old, most preferably, 5 day-old.

It will be appreciated that the stem cell, stem cell line or embryoid body of the present invention can be further differentiate into differentiated cells, tissue or even organs.

Such differentiated cells, tissue or organs can be used to develop disease models of various genetic disorders. For example, osteoblasts carrying mutations in the OSF2/CBFA1 gene can be used to study cleidocranial dysplasia (CCD, Lee B et al., Nat Genet. 1997; 16: 307-10); pancreatic cells carrying gain-of-function mutations in the cationic trypsinogen gene can be used to study hereditary pancreatitis (Tautermann G et al., Digestion. 2001; 64: 226-32); neuronal cells carrying mutations in the TATA box-binding protein gene can be used to study spinocerebellar ataxia type 17 (Bruni A C et al., Arch Neurol. 2004; 61: 1314-20); and mast cells carrying an activating mutation in c-kit which can be used to study mastocytosis (Dror Y et al., Br J Haematol. 2000; 108: 729-36).

Thus, according to another aspect of the present invention there is provided an isolated differentiated cell, tissue or organ carrying at least one disease-causing mutation in a genomic polynucleotide sequence thereof.

As used herein the phrase “differentiated cell” refers to any cell with a specialized function, shape and structure which can be derived from the stem cell, stem cell line or embryoid body of the present invention. Examples include, but are not limited to, neural cells, retina cells, epidermal cells, hepatocytes, pancreatic cells, osseous cells, cartilaginous cells, elastic cells, fibrous cells, myocytes, myocardial cells, bone marrow cells, endothelial cells, smooth muscle cells, and hematopoietic cells.

The phrase “tissue” refers to part of an organism consisting of an aggregate of cells having a similar structure and function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmunary tissue, gonadal tissue, hematopoietic tissue and fat tissue.

The phrase “organ” refers to a fully differentiated structural and functional unit in an animal that is specialized for some particular function. For example, head, brain, eye, leg, hand, heart, liver kidney, lung, pancreas, ovary, testis, and stomach.

The differentiated cell, tissue or organ of the present invention can be obtained by subjecting the stem cell, stem cell line or embryoid body to differentiation conditions. Such conditions may include withdrawing or adding nutrients, growth factors or cytokines to the medium, changing the oxygen pressure, or altering the substrate on the culture surface.

For example, embryonic stem cells can differentiate to osteoblasts (Bourne S. et al., Tissue Eng. 2004; 10: 796-806), hematopoietic cells (Kitajima K. Methods Enzymol. 2003; 365:72-83), vascular cells (Fraser S T., et al., Methods Enzymol. 2003; 365: 59-72), pancreatic precursors (Kahan B W et al., Diabetes. 2003; 52: 2016-24), neuronal precursors (Rathjen J, Rathjen P D. ScientificWorldJournal. March 2002 12; 2: 690-700), astrocytes (Tang F, et al., Cell Mol Neurobiol. 2002; 22: 95-101), and cardiac cells (Rolletschek A,. et al., 2004; Toxicol Lett. 149: 361-9; Foley A, and Mercola M, 2004; Trends Cardiovasc Med. 14: 121-5).

Following is a non-limiting description of a number of procedures and approaches for inducing differentiation of EBs to lineage specific cells.

Neural Precursor Cells

To differentiate the EBs of the present invention into neural precursors, four-day-old EBs are cultured for 5-12 days in tissue culture dishes including DMEM/F-12 medium with 5 mg/ml insulin, 50 mg/ml transferrin, 30 nM selenium chloride, and 5 mg/ml fibronectin (ITSFn medium, Okabe, S. et al., 1996, Mech. Dev. 59: 89-102). The resultant neural precursors can be further transplanted to generate neural cells in vivo (Brüstle, O. et al., 1997. In vitro-generated neural precursors participate in mammalian brain development. Proc. Natl. Acad. Sci. USA. 94: 14809-14814). It will be appreciated that prior to their transplantation, the neural precursors are trypsinized and triturated to single-cell suspensions in the presence of 0.1% DNase.

Oligodendrocytes and Myelinate Cells

EBs of the present invention can differentiate to oligodendrocytes and myelinate cells by culturing the cells in modified SATO medium, i.e., DMEM with bovine serum albumin (BSA), pyruvate, progesterone, putrescine, thyroxine, triiodothryonine, insulin, transferrin, sodium selenite, amino acids, neurotrophin 3, ciliary neurotrophic factor and Hepes (Bottenstein, J. E. & Sato, G. H., 1979, Proc. Natl. Acad. Sci. USA 76, 514-517; Raff, M. C., Miller, R. H., & Noble, M., 1983, Nature 303: 390-396]. Briefly, EBs are dissociated using 0.25% Trysin/EDTA (5 min at 37° C.) and triturated to single cell suspensions. Suspended cells are plated in flasks containing SATO medium supplemented with 5% equine serum and 5% fetal calf serum (FCS). Following 4 days in culture, the flasks are gently shaken to suspend loosely adhering cells (primarily oligodendrocytes), while astrocytes are remained adhering to the flasks and further producing conditioned medium. Primary oligodendrocytes are transferred to new flasks containing SATO medium for additional two days. Following a total of 6 days in culture, oligospheres are either partially dissociated and resuspended in SATO medium for cell transplantation, or completely dissociated and a plated in an oligosphere-conditioned medium which is derived from the previous shaking step [Liu, S. et al., (2000). Embryonic stem cells differentiate into oligodendrocytes and myelinate in culture and after spinal cord transplantation. Proc. Natl. Acad. Sci. USA. 97: 6126-6131].

Mast Cells

For mast cell differentiation, two-week-old EBs of the present invention are transferred to tissue culture dishes including DMEM medium supplemented with 10% FCS, 2 mM L-glutamine, 100 units/ml penicillin, 100 mg/ml streptomycin, 20% (v/v) WEHI-3 cell-conditioned medium and 50 ng/ml recombinant rat stem cell factor (rrSCF, Tsai, M. et al., 2000. In vivo immunological function of mast cells derived from embryonic stem cells: An approach for the rapid analysis of even embryonic lethal mutations in adult mice in vivo. Proc Natl Acad Sci USA. 97: 9186-9190). Cultures are expanded weekly by transferring the cells to new flasks and replacing half of the culture medium.

Hemato-Lymphoid Cells

To generate hemato-lymphoid cells from the EBs of the present invention, 2-3 days-old EBs are transferred to gas-permeable culture dishes in the presence of 7.5% CO₂ and 5% O₂ using an incubator with adjustable oxygen content. Following 15 days of differentiation, cells are harvested and dissociated by gentle digestion with Collagenase (0.1 unit/mg) and Dispase (0.8 unit/mg), both are available from F.Hoffman-La Roche Ltd, Basel, Switzerland. CD45-positive cells are isolated using anti-CD45 monoclonal antibody (mAb) M1/9.3.4.HL.2 and paramagnetic microbeads (Miltenyi) conjugated to goat anti-rat immunoglobulin as described in Potocnik, A. J. et al., (Immunology Hemato-lymphoid in vivo reconstitution potential of subpopulations derived from in vitro differentiated embryonic stem cells. Proc. Natl. Acad. Sci. USA. 1997, 94: 10295-10300). The isolated CD45-positive cells can be further enriched using a single passage over a MACS column Miltenyi).

It will be appreciated that since EBs are complex structures, differentiation of EBs into specific differentiated cells, tissue or organ may require isolation of lineage specific cells from the EBs.

Such isolation may be effected by sorting of cells of the EBs via fluorescence activated cell sorter (FACS) or mechanical separation of cells, tissues and/or tissue-like structures contained within the EBs.

Methods of isolating EB-derived-differentiated cells via FACS analysis are known in the art. According to one method, EBs are disaggregated using a solution of Trypsin and EDTA (0.025% and 0.01%, respectively), washed with 5% fetal bovine serum (FBS) in phosphate buffered saline (PBS) and incubated for 30 min on ice with fluorescently-labeled antibodies directed against cell surface antigens characteristics to a specific cell lineage. For example, endothelial cells are isolated by attaching an antibody directed against the platelet endothelial cell adhesion molecule-1 (PECAM1) such as the fluorescently-labeled PECAM1 antibodies (30884X) available from PharMingen (PharMingen, Becton Dickinson Bio Sciences, San Jose, Calif., USA) as described in Levenberg, S. et al., (Endothelial cells derived from human embryonic stem cells. Proc. Natl. Acad. Sci. USA. 2002. 99: 4391-4396). Hematopoietic cells are isolated using fluorescently-labeled antibodies such as CD34-FITC, CD45-PE, CD31-PE, CD38-PE, CD90-FITC, CD117-PE, CD15-FITC, class I-FITC, all of which IgG1 are available from PharMingen, CD133/1-PE (IgG1) (available from Miltenyi Biotec, Auburn, Calif.), and glycophorin A-PE (IgG1), available from Immunotech (Miami, Fla.). Live cells (i.e., without fixation) are analyzed on a FACScan (Becton Dickinson Bio Sciences) by using propidium iodide to exclude dead cells with either the PC-LYSIS or the CELLQUEST software. It will be appreciated that isolated cells can be further enriched using magnetically-labeled second antibodies and magnetic separation columns (WACS, Miltenyi) as described by Kaufman, D. S. et al., (Hematopoietic colony-forming cells derived from human embryonic stem cells. Proc. Natl. Acad. Sci. USA. 2001, 98: 10116-10721).

An example for mechanical isolation of beating cardiomyocytes from EBs is disclosed in U.S. Pat. Appl. No. 20030022367 to Xu et al. Briefly, four-day-old EBs of the present invention are transferred to gelatin-coated plates or chamber slides and are allowed to attach and differentiate. Spontaneously contracting cells, which are observed from day 8 of differentiation, are mechanically separated and collected into a 15-mL tube containing low-calcium medium or PBS. Cells are dissociated using Collagenase B digestion for 60-120 minutes at 37° C., depending on the Collagenase activity. Dissociated cells are then resuspended in a differentiation KB medium (85 mM KCl, 30 mM K₂HPO₄, 5 mM MgSO₄, 1 mM EGTA, 5 mM creatine, 20 mM glucose, 2 mM Na₂ATP, 5 mM pyruvate, and 20 mM taurine, buffered to pH 7.2, Maltsev et al., Circ. Res. 75:233, 1994) and incubated at 37° C. for 15-30 min. Following dissociation cells are seeded into chamber slides and cultured in the differentiation medium to generate single cardiomyocytes capable of beating.

It will be appreciated that the culturing conditions suitable for the differentiation and expansion of the isolated lineage specific cells include various tissue culture medium, growth factors, antibiotic, amino acids and the like and it is within the capability of one skilled in the art to determine which conditions should be applied in order to expand and differentiate particular cell types and/or cell lineages [reviewed in Fijnvandraat A C, et al., Cardiovasc Res. 2003; 58: 303-12; Sachinidis A, et al., Cardiovasc Res. 2003; 58: 278-91; Stavridis M P and Smith A G, 2003; Biochem Soc Trans. 31(Pt 1): 45-9].

As is mentioned hereinabove, the differentiated stem cell line or embryoid body of the present invention which carry the disease-causing mutation can be used to identify agents suitable for treating such genetic diseases.

Thus, according to another aspect of the present invention there is provided a method of identifying an agent suitable for treating a disorder associated with at least one disease-causing mutation.

As used herein “treating a disorder associated with at least one disease-causing mutation” refers to treating an individual suffering from a disorder such as a neurological disorder, a muscular disorder, a cardiovascular disorder, an hematological disorder, a skin disorder, a liver disorder, and the like that is caused by the disease-causing mutation of the present invention.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

The method is effected by subjecting cells of the stem cell line or the embryoid body of the present invention to differentiating conditions to thereby obtain differentiated cells exhibiting an effect of the at least one disease-causing mutation and exposing the differentiated cells to a plurality of molecules to identify at least one molecule (i.e., the agent) capable of regulating the effect of the at least one disease-causing mutation on the differentiated cells.

As used herein, “exposing the differentiated cells” refers to subjecting the differentiated cells of the present invention to various test molecules.

The phrase “cells exhibiting an effect of the at least one disease-causing mutation” refers to eukaryotic cells, preferably mammalian cells, more preferably, human cells, which include the disease-causing mutation in a genomic polynucleotide sequence thereof and which phenotype (i.e., structure and function) is effected by the disease-causing mutation. Such an effect can be a change in the size and shape of the cells and/or the cellular compartments (e.g., nucleus, cytoplasm, nucleolus), a change in receptor binding, cell secretion, intracellular reactions which lead to upregulation or downregulation of certain genes, a change in proliferation and/or differentiation processes of the cell, and the like.

Once the differentiated cells are obtained, the test molecules (e.g., drugs, minerals, vitamins, and the like) are applied on the differentiated cells and the structure and function of the cell is detected using the molecular, immunological and biochemical methods which are fully described hereinabove. Molecules which exert significant modulations of the structure and/or function of the differentiated cells become candidates for additional evaluations as suitable for treating the disorder associated with the disease-causing mutation of the present invention.

For example, to study the effect of abnormal repeat expansion of the CTG trinucleotide of the DMPK on mental retardation associated with Myotonic dystrophy neuronal cells can be expanded from EBs which are generated from the I-7 ES cell line (DM1) of the present invention. Briefly, four-day-old EBs are cultured under differentiating conditions [ITSFn medium, Okabe, 1996 (Supra)] and the resultant neuronal precursors can be tested for the activation of early (ERK1/2) and late (MAP2) differentiation markers, essentially as described in Quintero-Mora M L, et al. 2002; Biochem Biophys Res Commun. 295: 289-94.

To study the effect of a cystic fibrosis (CF) mutation on pancreas insufficiency associated with CF, ES cells carrying a CF mutation (e.g., N1303K) are subjected to pancreas precursor cell differentiation as described in [Kahan B W, 2003 (Supra)]. Briefly, ES cells are removed from their feeder layer cultures using 2 mmol/l EDTA containing 2% chicken serum. Following 7 days in suspension cultures intact EBs are plated onto gelatin-coated surfaces at a density of 30-50 EBs per 13-mm glass coverslip and are allowed to further differentiate for 1-5 weeks in high-glucose DMEM containing 10% FCS. The resulting pancreas precursors cells can be further compared to normal pancreas precursor cells with respect to gene expression patterns (e.g., insulin, glucagon, somatostatin, and pancreatic polypeptide) and cellular response to various drug molecules. For example, a drug molecule that will correct the abnormality of the apical membrane of the proximal duct epithelial cells which results in dehydrated protein-rich secretions from the proximal duct epithelial cells (Nousia-Arvanitakis S. J Clin Gastroenterol. 1999; 29: 138-42).

The effect of the disease-causing mutation on gene expression level can be determined using methods known in the art. Following is a non-limiting list of RNA-based methods which can be used according to the method of the present invention.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Oligonucleotide microarray—In this method oligonucleotide probes capable of specifically hybridizing with specific polynucleotide sequences are attached to a solid surface (e.g., a glass wafer). Each oligonucleotide probe is of approximately 20-25 nucleic acids in length. To compare the expression pattern of such polynucleotides in cells harboring a disease-causing mutation vs. control cells, RNA is preferably extracted from the cells, cell lines, embryoid bodies, tissue or organs of the present invention using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Although cell profiling methods which analyze the transcriptome of the cells of the present invention are preferred for their accuracy and high throughput capabilities, it will be appreciated that the present invention can also utilize protein analysis tools for profiling the cells of the cultures.

Expression and/or activity level of proteins expressed in the cells of the cultures of the present invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

It will be appreciated that large-scale proteomic analysis can be also employed in order to identify biomarkers associated with the disease-causing mutations of the present invention. For example, the proteins of the cells, cell lines, embryoid bodies, tissues or organs of the present invention can be subjected to various dissolving agents (e.g., SDS, Urea) followed by determination of protein sequencing or mass spectrometry analysis. Thus, the stem cell, stem cell line, embryoid body, differentiated cell, tissue or organ of the present invention which carry a disease-causing mutation can be used for drug discovery and testing, cell-based therapy, transplantation, production of biomolecules, testing the toxicity and/or teratogenicity of compounds and facilitating the study of developmental and other biological processes.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N. Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Generation of Human Embryonic Cell Lines Harboring Genetic Mutations

Human ES cell lines were generated from discarded embryos blastocysts following preimplantation genetic diagnosis (PGD) and the presence of the disease-causing-mutations in the ESCs was determined, as follows.

Materials and Experimental Methods

Blastocyst cultivation—In vitro fertilization was performed by sperm injection (ICSI) into oocytes retrieved following gonadotrophin-induced ovarian stimulation. Injected oocytes (18-19 hours post-ICSI) were monitored for the presence of pronuclear formation and zygotes with normal pronucleai were transferred (as drops under oil) for blastocyst cultivation in the presence of the Cook growth medium [specialized Cook media for insemination (IM), growth (GM) and blastocyst development (BM), Queensland, Australia].

Seventy-six discarded embryos were donated by the PGD program at the Rambam Medical Center; the donor couples signed consent forms which were approved by the hospital and national health committee. The donated embryos were either embryos that underwent PGD with unclear results whose parents decided to not retrieve and/or with positive identification of disease-causing-mutations, or were found unsuitable for embryo transfer according to the IVF grading.

Micromanipulation blastomere biopsy—Blastomeres having 6-8 cells on the third day in culture were subjected to a blastomere biopsy, as follows. Each embryo was gently held by a holding micropipette (20 micron diameter aperture) and the zona pellucida was drilled using an aperture micropipette (10-micron in diameter) filled with acid Tyrode's solution (pH 2.4; Sigma Chemical Co., St. Louis, Mo., USA). The resulting opening of the zona pellucida was slightly smaller than the size of the blastomere (˜40 microns). A 40-micron micropipette filled with PBS was inserted through the opening, and the nearest blastomere(s) was aspirated. For genetic analysis, each of the aspirated blastomere's cell was transferred to a PCR tube.

Pre-implantation genetic diagnosis (PGD)—Prior to PCR amplification, the selected blastomere cell was lysed for one hour at 37° C. using 2 μl-of 125 μg/ml PCR grade proteinase K (Roche Diagnostic GmbH, Mannheim, Germany) and 1 μA of 17 μM SDS (Sigma Chemical Co., St. Louis, Mo., USA), prepared in nuclease free water (Promega, Madison Wis.). The proteinase K reaction was stopped by heat inactivation (15 minutes at 95° C.) and the PCR mixture was added directly to the cell lyzate. The first PCR was performed by adding a 17 μl PCR reaction mixture to the cell lyzate and the nested PCR was performed by adding 2 μl of the first PCR product into 18 μl of the nested PCR reaction mixture, to reach a final volume of 20 μl in each case. PCR reactions included initial denaturation for 5 minutes, followed by 35 cycles of denaturation (at 95° C. for first PCR, or 94° C. for nested PCR), annealing (at the noted annealing temperature in Table 1, hereinbelow) and elongation (at 72° C.), for 30 seconds each, and a final elongation for 7 minutes at 72° C. PCR primers and conditions are listed in Table 1, hereinbelow. Nested PCR products were separated on a 3% nusieve agarose (Biowhittaker Molecular Applications, Rockland, Me. USA) and photographed under UV illumination.

TABLE 1
PCR primers and conditions for genetic diagnosis

| Disorder | Forward (F) and reverse (R) | Composition of PCR | Anneal. |
|---|---|---|---|
| (Gene) | primers (SEQ ID NO:) | reaction mixture | Temp. |
| Myotonic | First PCR: | 1 IU BioTaq polymerase | 65° C. |
| Dystrophy | F (101): | and 1 × PCR buffer | |
| (DMPK) | 5′-CTTCCCAGGCCTGCAGTTTGCCCATC | (Bioline), 10% DMSO, | |
| GenBank | (SEQ ID NO:1) | 2 mM MgCl₂, 0.2 mM dNTP | |
| Accession No. | R (102): | and 2 pmole of each of | |
| NM_004409 | 5′-GAACGGGGCTCGAAGGGTCCTTGTAGC | the primers | |
| | (SEQ ID NO:2) | | |
| | Nested PCR | 1 IU Taq polymerse and | 65° C. |
| | F (409): | 1 × PCR buffer (Qiagen | |
| | 5′-GAAGGGTCCTTGTAGCCGGGAA | GmbH, Hilden, Germany), | |
| | (SEQ ID NO:3) | 1.5 mm MgCl₂, 0.2 mM | |
| | R (410): | dNTP, Q-solution | |
| | 5′-GGGATCACAGACCATTTCTTTCT | (Qiagen) and 2 pmole of | |
| | (SEQ ID NO:4) | each of the PCR primers; | |
| Van | First PCR | 1 IU BioTaq polymerase | 60° C. |
| Waardenburg | F: 5′-CTTCCCACAGTGTCCACTCC | and 1 × PGR buffer | |
| syndrome | (SEQ ID NO:5) | (Bioline), 1.5 mM MgCl₂, | |
| (PAX3) | R: 5′-GAGGATTGCAAGGCTTATGG | 0.2 mM dNTP, 2 pmole of | |
| GenBank | (SEQ ID NO:6) | each of the PCR primers | |
| Accession No. | Nested PCR | 1 IU Taq polymerse and | 60° C. |
| NM_000438 | F: 5′-ACGGCAGGCCGCTGCCCAAC | 1 × PCR buffer (Qiagen), | |
| | (SEQ ID NO:7) | 1.5 mM MgCl₂, 0.2 mM | |
| | R: 5′-AGTCTGGGAGCCAGGAG | dNTP, Q-solution | |
| | (SEQ ID NO:8) | (Qiagen) and 2 pmole of | |
| | | each of the PCR primers | |
| Cystic | F (w1): | 1 IU Taq polymerse and | 60° C. |
| Fibrosis | 5′-TACCTATATGTCACAGAAGT | 1 × PCR buffer (Qiagen | |
| (CFTR) | R (w2): | GmbH, Hilden, Germany), | |
| GenBank | 5′-GTACAAGTATCAAATAGCAG | 1.5 mM MgCl₂, 0.2 mM | |
| No. | | dNTP, Q-solution | |
| M28668 | | (Qiagen) and 2 pmol of | |
| | | each of the PCR primers | |
| | Following PCR the fragment | | |
| | (270 bp long) is subjected | | |
| | to restriction enzyme | | |
| | analysis using the MnII | | |
| | restriction enzyme. | | |
| metachromatic | First PCR F (2098): | 1 IU Taq polymerse and | 60° C. |
| leukodystrophy | 5′-GCAGTCTCTCTTCTTCTAGC | 1 × PCR buffer (Qiagen | |
| (Arylsulfatase | R (2264): | GmbH, Hilden, Germany), | |
| A) GenBank No. | 5′-AGGGGCCAGGGATCTAGGGC | 1.5 mM MgCl₂, 0.2 mM | |
| AY271820 | | dNTP, Q-solution | |
| | | (Qiagen) and 2 pmole of | |
| | | each of the PCR primers | |
| | Following PCR the fragment | | |
| | is subjected to restriction | | |
| | enzyme analysis using the | | |
| | AluI restriction enzyme. | | |

Derivation of hES cell lines—After digestion of the zona pellucida by Tyrode's acidic solution (Sigma, St Louis, Mo., USA) or its mechanical removal, the exposed blastocysts were placed on mitotically inactivated mouse embryonic fibroblast (MEF) feeder layers in the presence of a culture medium consisting of 80% KO-DMEM, 1 mM L-glutamine, 0.1 mM β-mercaptoethanol, 1% non-essential amino acid stock (all from Gibco Invitrogen corporation products, San Diego, Calif., USA products) and supplemented with 20% defined FBS (HyClone, Utah, USA), Following 5-10 days in culture, the intracellular mass (ICM) of the expanded blastocyst was excised (using a needle and a micropipettor) and transferred to fresh MEF covered plates. The pluripotent cells (derived from the ICM) were further cultured in the presence of the same culture medium and passaged every 4-10 days, depending on the cell density.

Culture of hES cells—From passage 7-10 and onward, the cells were cultured on MEFs covered plates using a culture medium consisting of 85% KO-DMEM, 1 mM L-glutamine, 0.1 mM β-mercaptoethanol, 1% non-essential amino acid stock, 4 ng/ml basic fibroblast growth factor and supplemented with 15% ko-serum replacement and were routinely passaged every four to six days using 1 mg/ml type IV Collagenase (All products from Gibco Invitrogen). For storage, the cells were frozen in liquid nitrogen using a freezing solution consisting of 10% DMSO (Sigma), 10% FBS (Hyclone) and 80% KO-DMEM.

PCR analysis of human ES cell lines—DNA was extracted from the ES cell lines using the Genomic DNA isolation kit (Wizard, Promega, Madison, Wis., USA) according to the manufacturer's instructions and 2 μl of genomic DNA was employed for PCR analysis using the PCR primers and conditions listed in Table 1, hereinabove.

Karyotype analysis—Karyotype analysis was performed as previously described (Amit et al, 2003). ES cells metaphases were blocked using colcemid CaryoMax colcemid solution, Invitrogen, Grand island, N.Y., USA) and nuclear membranes were lysed in an hypotonic solution according to standard protocols (International System for Human Cytogenetic Nomenclature, ISCN). G-banding of chromosomes was performed according to manufacturer's instructions (Giemsa, Merck). Karyotypes of at least 20 cells per sample were analyzed and reported according to the ISCN.

Immunohistochemistry—Human ES cells were fixed for 15 minutes in 4% paraformaldehyde, blocked for 20 minutes in 2% normal goat serum in PBS and incubated for overnight at 4° C. with 1:20 dilutions of SSEA1, SSEA3, SSEA4, TRA1-60, TRA1-81 mouse anti-human antibodies, provided by Prof. P Andrews the University of Sheffield, England. Cells were then washed in PBS and further incubated with 1:100 dilutions of Donkey anti-mouse IgG antibodies conjugated to the fluorochrome Cys 3 (Chemicon International, Temecula Calif., USA). Cells were visualized under an inverted fluorescent microscope (Inverted fluorescent microscope, CARL Zeiss, Germany).

Experimental Results

Pre-implantation genetic diagnosis (PGD) identified blastocyst cells harboring various disease-causing-mutations—To determine the presence or absence of disease-causing-mutations of the Van Waardenburg (WS1), Myotonic Dystrophy (DM1), cystic fibrosis (CF) or metachromatic leukodystrophy (MLD), PGD was performed on single cell's DNA (derived from a blastocyst) using PCR primers specific to the PAX3 (GenBank Accession No. NM_{—}000438), DMPK (GenBank Accession No. NM-004409), CFTR (GenBank Accession No. M28668), or Arylsulfatase A (GenBank Accession No. AY271820), respectively (data not shown).

Generation of ES cell lines from blastocysts—Out of the 76 discarded embryos, 31 were developed to the blastocyst stage. For ES cell lines isolation, the embryos were plated as a whole blastocyst on MEFs (FIG. 1a). Following 5-10 days in culture, the ICM outgrowth was detected in 5/31 embryos (FIG. 1b) and the pluripotent stem cells (isolated from the ICM) were transferred to MEF covered plates for further culturing.

Genetic analysis reveals the presence of the Van Waardenburg syndrome (WS) disease-causing-mutation in a human ES cell line—In order to determine if cells of a human ES cell line which was derived from an IVF-blastocyst of a known Van Waardenburg family (family BU-53) carry a WS disease-causing-mutation, the DNA was subjected to PCR analysis using the PAX3-specific PCR primers (SEQ ID NOs:5-8). As is shown in FIG. 2a, while DNA of a normal (i.e., unaffected) individual revealed a single band of 100 bp, the DNA of the affected parent and the resultant human ES cell line, each exhibited two bands of 100 and 100-28 bp, corresponding to the wild-type allele and the 28 bp—deleted allele, respectively. Sequence analysis of the 100-28 allele confirmed the presence of a 28 bp deletion at the 3′-end of exon 2 in the affected parent and the 1-5 (WS1) ES cell line. The deletion sequence corresponds to nucleic acid coordinates 54129-54157 of GenBank Accession No. AC010980 which includes the genomic sequence of PAX3, to nucleic acid coordinates 510-538 of GenBank Accession No. X15043 (SEQ ID NO:34) which includes part of the gene encoding PAX3, and in part (due to an exon boundary) to nucleic acid coordinates 662-682 of GenBank Accession No. NM_{—}000438 (SEQ ID NO:23) which includes the full length mRNA encoding PAX3.

Genetic analysis reveals the presence of the Myotonic Dystrophy (DM)—disease-causing-mutation in a human ES cell line—DNA extracted from cells of a human ES cell line (I-7) which was derived from an IVF-blastocyst of a known DM family was subjected to PCR analysis using the DM specific primers (SEQ ID NOs:1-4). As is shown in FIG. 2b, when the PCR products were electrophoresed (using an 8% polyacrylamide gel) and stained [using silver staining (Lerer I, et al., 1994, Am. J. Med. Gen. 52: 79-84)], abnormal expansions of the CTG repeats were observed in the DNA of the 1-7 (DM1) human ES cell line (1.4 and 3.0 Kb), as well as in DNA of several DM-affected individuals.

Human ES cell lines harbor the cystic fibrosis or metachromatic leukodystrophy disease-causing-mutations—The J-3 or the I-8 and I-9 ES cell lines were found to carry, in a heterozygous form, the W1282X or P377L (1505C→T in GenBank Accession No. NM_{—}000487, SEQ ID NO:21) genetic mutations which cause cystic fibrosis or metachromatic leukodystrophy (MLD), respectively (data not shown).

Human ES cells harboring genetic mutations exhibit normal characteristics of human ES cell lines—The I-7 (DM1) and I-5 (WS1) ES cell lines harboring the myotonic dystrophy and Van Waardenburg syndrome disease-causing mutations, respectively, demonstrated colony and cell morphology which are typical of human ES cell lines, i.e. round colonies with clear borders, spaces between cells, high cytoplasm to nucleus ratio and existence of two to four nucleoli (FIGS. 1c-d). In addition, as is shown in FIGS. 3a-f, immunohistochemistry staining of the I-5 (WS1) ESCs using clonal primary antibodies for undifferentiated surface markers revealed negative staining for stage-specific embryonic antigen (SSEA)-1, weak or no staining for SSEA3, and positive staining for SSEA4, tumor recognition antigen (TRA)-1-60 and TRA-1-81 as previously shown for human ES cell lines (Thomson at el, 1998; Reubinoff et al, 2000). Similar results were obtained with the I-7 (DM1) ESCs following 37 passages (not shown). Moreover, karyotype analysis which was conducted on cells at passage 30 and 17 for the I-5 (SW1) and I-7 (DM1) cell lines, respectively, revealed a normal 46, XX karyptypes in at least 40 cells in each case.

Thus, these results demonstrate for the first time, the generation of human ES cell lines harboring disease-causing-mutations of the Van Waardenburg syndrome, Myotonic Dytrophy, cystic fibrosis or metachromatic leukodystrophy. Such human ES cell lines can be used for studying the molecular and physiological pathways leading to such genetic disorders and in developing suitable treatments for such disorders.

### Example 2

### Embryoid Bodies and Teratomas can be Generated from Human ES Cell Lines Harboring Disease-Causing-Mutations

To further test the suitability of human ES cell lines harboring disease-causing-mutations to differentiate into all three embryonic germ layers, ES cell lines were transferred to suspension culture or were injected into SCID mice, and the expression pattern of several differentiation markers was determined in the resulting embryoid bodies or teratomas, respectively.

Materials and Experimental Methods

Immunohistochemistry—was performed as described in Example 1, hereinabove.

EB formation—ES cells from four to six confluent wells (40-60 c²m) were collected using 1 mg/ml type IV Collagenase (Invitrogen), further broken into small clumps using 1000 μl Gilson pipette tips, and cultured in suspension in 58-mm Petri dishes (Greiner, Germany). EBs were grown in 80% KO-DMEM, 1 mM L-glutamine, 0.1 mM β-mercaptoethanol, 1% non-essential amino acid stock (all from Gibco Invitrogen) and supplemented with 20% defined FBS (HyClone).

Teratoma formation—Cells from six confluent wells of a six-well plate (60 c²m) were harvested and injected into the rear leg muscle of four-week-old male SCID-beige mice (Harlan, Jerusalem Israel). Resulting teratomas were examined histologically, at least 12 weeks post-injection. Briefly, teratomas were fixed in 10% neutral-buffered formalin, dehydrated in graduated alcohol (70%-100%) and embedded in paraffin. For histological examination, 1-5 μm sections were deparafinized and stained with hematoxylin/eosin (H&E).

RT-PCR—Total RNA was isolated from either undifferentiated cells grown for 34 and 41 passages post derivation, or from 10 day-old EBs using Tri-Reagent (Sigma, St. Louis, Mo.), according to the manufacturer's protocol. cDNA synthesis was performed from 1 μg total RNA using MMLV reverse transcriptase RNase H minus (Promega, Madison, Wis., USA). PCR reactions included an initial strand denaturation for 5 minutes at 94° C. followed by repeated cycles of denaturation (94° C. for 30 seconds), annealing at the noted temperatures (see Table 1, hereinbelow) for 30 seconds and elongation at 72° C. for 30 seconds. PCR primers and reaction conditions used are described in Table 2, hereinbelow. PCR products were size-fractionated using 2% agarose gel electrophoresis.

TABLE 2

RT-PCR primers and conditions for the identification
of embryonic germ layer specific markers

| Gene product | | | | |
|---|---|---|---|---|
| (Accession | | Forward (F) and reverse (R) | Reaction | Size |
| number) | SEQ ID NOs. | primers (5′→3′) | Condition | (bp) |
| Oct-4 | SEQ ID NO:9 | F: GAGAACAATGAGAACCTTCAGGA | 30 cycles | 219 |
| (S81255) | SEQ ID NO:10 | R: TTCTGGCGCCGGTTACAGAACCA | at 60° C. | |
| | | | in 1.5 mM | |
| | | | MgCl₂ | |
| Albumin | SEQ ID NO:11 | F: TGCTTGAATGTGCTGATGACAGGG | 35 cycles | 302 |
| (AF542069) | SEQ ID NO:12 | R: AAGGCAAGTCAGCAGCCATCTCAT | at 60° C. | |
| | | | in 1.5 mM | |
| | | | MgCl₂ | |
| α-fetoprotein | SEQ ID NO:13 | F: GCTGGATTGTCTGCAGGATGGGGAA | 30 cycles | 216 |
| (BC027881) | SEQ ID NO:14 | R: TCCCCTGAAGAAAATTGGTTAAAAT | at 60° C. | |
| | | | in 1.5 mM | |
| | | | MgCl₂ | |
| NF-68KD | SEQ ID NO:15 | F: GAGTGAAATGGCACGATACCTA | 30 cycles | 473 |
| (AY1566990) | SEQ ID NO:16 | R: TTTCCTCTCCTTCTTCACCTTC | at 60° C. | |
| | | | in 2 mM | |
| | | | MgCl₂ | |
| α-cardiac | SEQ ID NO:17 | F: GGAGTTATGGTGGGTATGGGTC | 35 cycles | 486 |
| actin | SEQ ID NO:18 | R: AGTGGTGACAAAGGAGTAGCCA | at 65° C. | |
| (NM_005159) | | | in 2 mM | |
| | | | MgCl₂ | |
| β-actin | SEQ ID NO:19 | F: ATCTGGCACCACACCTTCTACAATGAGCTGCG | 35 cycles | 838 |
| (NM_001101) | SEQ ID NO:20 | R: CGTCATACTCCTGCTTGCTGATCCACATCTGC | at 62° C. | |
| | | | in 1.5 mM | |
| | | | MgCl₂ | |

Experimental Results

ES cells harboring disease-causing-mutations spontaneously differentiate into the three embryonic germ layer cell types in vitro—To verify that human ES cells harboring disease-causing-mutations are functionally, as well as phenotypically consistent with normal human ES cells, ES cell were removed from their feeder layers and were cultured in suspension. As is shown in FIGS. 4a and b, both the I-7 (DM1) and the I-5 (WS1) ES cell lines, respectively, spontaneously formed embryoid bodies (EBs) including cystic EBs.

The functionality of the isolated EBs was further tested by IHC using various embryonic cell markers. As is further shown in FIGS. 4c-f, EBs expressed nestin which is derived from an ectodermal origin, insulin, which is from a endodermal origin, and troponin, a marker of the mesodermal origin. These results demonstrate that the ES cell lines harboring disease-causing-mutations are capable of differentiating into all three embryonic germ layers, i.e., mesoderm, endoderm and ectoderm.

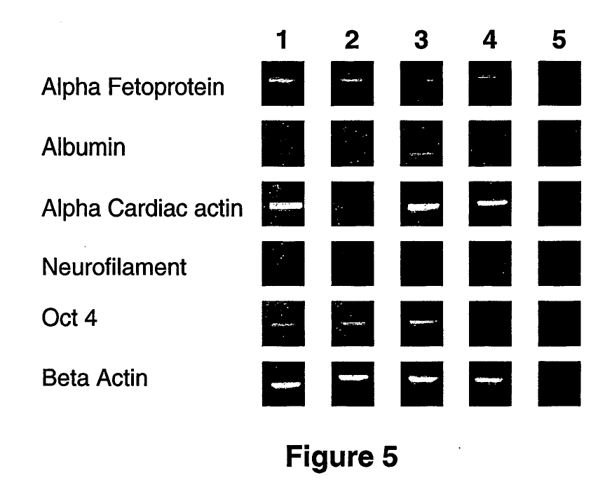

ES-consistent gene expression within the EBs was further verified using RT-PCR. As shown in FIG. 5, while undifferentiated cells expressed high levels of Oct 4, a marker for pluripotent embryonic stem and germ cells (Pesce M, and Scholer H R., 2001, Stem Cells 19: 271-8), cells harvested from five-day-old EBs expressed genes, which are associated with cellular differentiation including neurofilament (NF-68 kD) which is related with embryonal ectoderm, α-cardiac actin which is associated with embryonal mesoderm, and albumin which is associated with embryonal endoderm. The diminished Oct 4 expression in the EB sample obtained from the DM1 ES cell line was consistent with previous reports of diminished Oct 4 expression following differentiation of totipotent cells to somatic lineages (Thomson J A, et al., 1998, Science 282: 1145-7; Reubinoff B E, et al., 2000, Nat. Biotechnol. 18: 399-404). As have previously reported elsewhere (Schuldiner M. et al., 2000, Proc Natl Acad Sci USA 97: 11307-12; Amit, M. et al., 2003, Biol. Reprod. 68: 2150-2156; Kehat, I. et al., 2001, J Clin Invest 108: 407-14) ES cell cultures might have some degree of background differentiation. Indeed, some of the cell-specific genes, like α-fetoprotein, albumin and a-cardiac actin, were also expressed in the undifferentiated ES cells (FIG. 5, lanes 1 and 2).

Thus, these results demonstrate that human ES cells harboring disease-causing-mutations are capable of creating functional EBs consisting of all three embryonic germ layers.

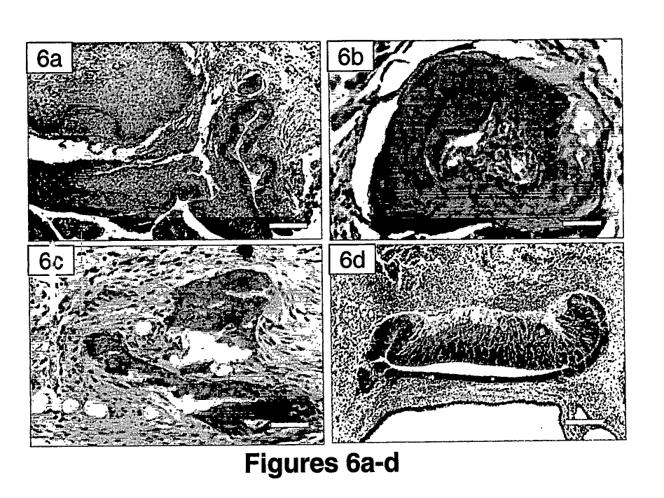

Human ES cells harboring disease-causing-mutations differentiate into embryonic germ layers in vivo—To further substantiate the ability of human ES cells harboring disease-causing-mutations to differentiate into embryonal germ layers, ES cells were tested for teratoma formation in vivo. Following injection into the hindlimb muscle of SCID Beige mice, the I-7 (DM1) and I5 (WS1) ES cells were able to form teratomas. As is shown in FIGS. 6a-d, each teratoma contained representative tissues of the three embryonic germ layers, including cartilage and muscle tissue of the mesodermal origin, gut-like epithelium of the endodermal origin, and nerve tissue which is of the ectodermal origin.

In conclusion, human ES cells harboring disease-causing-mutations such as those causing myotonic dystrophy and Van Waardenburg syndromes exhibit phenotypic as well as functional characteristics of ES cell line. Following their differentiation in vitro (i.e., into EBs) and in vivo (i.e., in teratomas), ES cells expressed genes associated with all three embryonal germ layers.

Discussion

The pluripotency and immortality of hES cells may be utilized for the development of research models for genetic diseases such as DM and WS. The ability of ES cells to differentiate into any cell type of the adult human body can facilitate in understanding the processes affecting each system. For example, directed differentiation of human ES cells carrying disease-causing-mutations into cardiomyocytes and/or stratified muscle (for DM), or nerve and/or pigment producing cells (for WS), may prove invaluable for understanding the pathogenesis of these diseases. For some of these differentiation models, directing protocols for human ES already exist (Xu et al, 2002; Mummery et al, 2002; Reubinoff et al, 2001; Zhang et al, 2001). Such differentiation models can be also used for in vitro drug testing.

In addition, the ES cell lines of the present invention can be used to monitor the effect of the mutation during differentiation. For example, the role of PAX3 in early nerve development and the evolution of the (CTG)n repeats characterizing DM during continuous culturing of ES cells.

Gene therapy is often based on targeted correction, using small fragments of a corrected region of the gene (Colosimo et al, 2001). The availability of human ES cell lines harboring disease-causing-mutations such as the W1282X in the CFTR gene (causing cystic fibrosis) and the P377L (1505C→T in GenBank Accession No. NM_{—}000487 SEQ ID NO:21) in the Arylsulfatase A gene (causing metachromatic leukodystrophy) would benefit the development of targeted correction models for these mutations.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References are Cited in the Text

1. Amit M, Itskovitz-Eldor J. (2002). Derivation and spontaneous differentiation of human embryonic stem cells. J. Anat. 200, 225-232.

2. Amit M, Margulets V, Segev H, Shariki C, Laevsky I, Coleman R, and Itskovitz-Eldor J. Human feeder layers for human embryonic stem cells. Biol Reprod 2003; 68:2150-2156.

3. Asher J H Jr, Harrison R W, Morell R, Carey M L, Friedman T B. Effects of Pax3 modifier genes on craniofacial morphology, pigmentation, and viability: a murine model of Waardenburg syndrome variation. Genomics. 1996; 34:285-298.

4. Baldwin C T, Hoth C F, Amos J A, da-Silva E O, Milunsky A. An exonic mutation in the HuP2 paired domain gene causes Waardenburg's syndrome. Nature 1992; 355:637-638.

5. Brook J D, McCurrach M E, Harley H G, Buckler A J, Church D, Aburatani H, Hunter K, Stanton V P, Thirion J P, Hudson T, et al. Molecular basis of myotonic dystrophy: expansion of a trinucleotide (CTG) repeat at the 3′ end of a transcript encoding a protein kinase family member. Cell. 1992; 68:799-808.

6. Colosimo A, Goncz K K, Novelli G, Dallapiccola B, Gruenert D C. Target correction of a defective selectable marker gene in human epithelial cells by small DNA fragments. Molecular Therapy 2001; 178-185.

7. Fraser G R. The causes of profound deafness in childhood. Baltimore: Johns Hopkins University Press 1976.

8. Itskovitz-Eldor J, Schuldiner M, Karsenti D, Eden A, Yanuka O, Amit M, Soreq H, Benvenisty N. Differentiation of human embryonic stem cells into embryoid bodies comprising the three embryonic germ layers. Mol Med 2000; 6:88-95.

9. Lerer I, Meiner V, Pashut-Lavon I, Abeliovich D. Molecular diagnosis of Prader-Willi syndrome: Parent-of-origin dependent methylation sites and non-isotopic detection of (CA), dinucleotide repeat polymorphisms. Am. J Med. Gen. 1994; 52: 79-84.

10. Liquori C L, Ricker K, Moseley M L et al. Myotonic dystrophy type 2 caused by CCTG expansion in intron 1 of ZNF9. Science 2001; 293: 864-867.

11. Mahadevan M, Tsilfidis C, Sabourin L, Shutler G, Amemiya C, Jansen G, Neville C, Narang M, Barcelo J, O'Hoy K, et al. Myotonic dystrophy mutation: an unstable CTG repeat in the 3′ untranslated region of the gene. Science 1992 255:1253-1255.

12. Mankodi A, Thornton C A. Myotonic syndromes. Curr. Opi. Neurol. 2002; 15:545-552.

13. McKusick V M. Mendelian inheritance in man. 10th John Hopkines University press, Baltimor, 1992.

14. Meola G. Clinical and genetic heterogeneity in myotonic dystrophies. Muscle Nerve 2000; 23: 1789-1799.

15. Mitalipova M, Calhoun J, Shin S, Wininger D, Schulz T, Noggle S, Venable A, Lyons I, Robins A, Stice S. Human Embryonic Stem Cell Lines Derived from Discarded Embryos. Stem Cells 2003; 21:521-526.

16. Mummery, C., Ward, D., van den Brink, C. E., Bird, S. D., Doevendans, P. A, Opthof, T., Brutel de la Riviere, A., Tertoolen, L., van der Heyden, M., Pera M. Cardiomyocyte differentiation of mouse and human embryonic stem cells. J. Anat. 2002; 200: 233-242.

17. Sermon K, Lissens W, Joris H, Seneca S, Desmyttere S, Devroey P, Van Steirteghem A, Liebaers I. Clinical application of preimplantation diagnosis for myotonic dystrophy. Prenat Diagn. 1997; 17:925-932.

18. Reubinoff B E, Pera M F, Fong C, Trounson A, Bongso A. Embryonic stem cell lines from human blastocysts: somatic differentiation in vitro. Nat Biotechnol 2000; 18:399-404.

19. Reubinoff, B. E., Itsykson, P., Turetsky, T., Pera, M. F., Reinhartz, E., Itzik, A., Ben-Hur, T. Neural progenitors from human embryonic stem cells. Nat. Biotechnol. 2001; 19: 1134-1140.

20. Tassabehji M, Read A P, Newton V E, Harris R, Balling R, Gruss P, Strachan T. Waardenburg's syndrome patients have mutations in the human homologue of the Pax-3 paired box gene. Nature 1992; 355:635-636.

21. Thomson J A, Itskovitz-Eldor J, Shapiro S S, Waknitz M A, Swiergiel J J, Marshall V S, Jones J M. Embryonic stem cell lines derived from human blastocysts. Science 1998; 282:1145-7 [erratum in Science 1998;282:1827].

22. Tian B, White R J, Xia T, Welle S, Turner D H, Mathews M B, Thornton C A. Expanded CUG repeat RNAs form hairpins that activate the double-stranded RNA-dependent protein kinase PKR. RNA 2000; 6:79-87.

23. Waardenburg P J. A new syndrome combining developmental anomalies of the eyelids, eyebrows and nose root with pigmentary defects of the iris and head hair and with congenital deafness. Am J. Hum Genet 1951; 3:195-253.

24. Xu, C., Police, S., Rao, N., Carpenter, M. K. Characterization and enrichment of cardiomyocytes derived from human embryonic stem cells. Circ. Res. 2002; 91: 501-508.

25. Zhang, S-C., Wernig, M., Duncan, I. D., BrÜstle, O., Thomson, J. A. In vitro differentiation of transplantable neural precursors from human embryonic stem cells. Nat. Biotechnol. 2001; 19: 1129-1133.

1-51. (canceled)

52. An isolated stem cell or stem cell line carrying a disease-causing mutation in a genomic polynucleotide sequence thereof.

53. The isolated stem cell or stem cell line of claim 52, wherein said stem cell is of embryonic origin.

54. The isolated stem cell or stem cell line of claim 52, wherein said stem cell is of human origin.

55. The isolated stem cell or stem cell line of claim 52, wherein said disease-causing mutation is selected from the group consisting of a missense mutation, a nonsense mutation, a frameshift mutation, a readthrough mutation, a promoter mutation, a regulatory mutation, a deletion, an insertion, an inversion, a splice mutation and a duplication.

56. The isolated stem cell or stem cell line of claim 52, wherein said disease-causing mutation is selected from the group consisting of a missense mutation, a nonsense mutation, a frameshift mutation, a readthrough mutation, a promoter mutation, a regulatory mutation, a deletion, an insertion, an inversion, a splice mutation and a duplication.

57. The isolated stem cell or stem cell line of claim 52, wherein said disease-causing mutation is selected from the group consisting of the W1282X as set forth in SEQ ID NO:24 associated with cystic fibrosis, the PAX3-del28 (510del28 in SEQ ID NO:34) associated with van Waardenburg syndrome, more than 50 (CTG) repeats as set forth in SEQ ID NO:22 associated with Myotonic dystrophy and the 1505C→T (P377L) as set forth in SEQ ID NO:21 associated with metachromatic leukodystrophy.

58. The isolated stem cell or stem cell line of claim 52, wherein said stem cell is capable of being maintained in an undifferentiated state for at least 41 passages.

59. The isolated stem cell or stem cell line of claim 52, wherein said stem cell exhibits a karyotype of 46, XX or 46, XY following at least 30 passages.

60. The isolated stem cell or stem cell line of claim 52, wherein said stem cell exhibits pluripotent capacity following 40 passages.

61. An isolated embryoid body comprising a plurality of cells at least some of which carry a disease-causing mutation in a genomic polynucleotide sequence thereof.

62. The isolated embryoid body of claim 61, wherein said embryoid body is derived from a stem cell or a stem cell line.

63. The isolated embryoid body of claim 62, wherein said stem cell is of human origin.

64. The isolated stem cell or stem cell line of claim 62, wherein said stem cell exhibits a karyotype of 46, XX or 46, XY following at least 30 passages.

65. The isolated embryoid body of claim 61, wherein said disease-causing mutation is selected from the group consisting of a missense mutation, a nonsense mutation, a frameshift mutation, a readthrough mutation, a promoter mutation, a regulatory mutation, a deletion, an insertion, an inversion, a splice mutation and a duplication.

66. The isolated embryoid body of claim 61, wherein said disease-causing mutation is associated with a genetic disorder selected from the group consisting of cystic fibrosis (CF), myotonic dystrophy (DM), van Waardenburg syndrome (WS), metachromatic leukodystrophy (MLD), Gorlin disease, Huntington's disease (HD), spinal muscular atrophy (SMA) and Duchenne muscular dystrophy (DMD).

67. The isolated embryoid body of claim 61, wherein said disease-causing mutation is selected from the group consisting of the W1282X as set forth in SEQ ID NO:24 associated with cystic fibrosis, the PAX3-del28 (510del28 in SEQ ID NO:34) associated with van Waardenburg syndrome, more than 50 (CTG) repeats as set forth in SEQ ID NO:22 associated with Myotonic dystrophy and the 1505C→T (P377L) as set forth in SEQ ID NO:21 associated with metachromatic leukodystrophy.

68. The isolated embryoid body of claim 61, wherein said embryoid body is capable of differentiating into cells of the embryonic ectoderm, embryonic endoderm and/or embryonic mesoderm.

69. An isolated differentiated cell, tissue or organ carrying at least one disease-causing mutation in a genomic polynucleotide sequence thereof.

70. The isolated differentiated cell, tissue or organ of claim 69, wherein said differentiated cell, tissue or organ is of human origin.

71. The isolated differentiated cell, tissue or organ of claim 69, wherein said disease-causing mutation is selected from the group consisting of a missense mutation, a nonsense mutation, a frameshift mutation, a readthrough mutation, a promoter mutation, a regulatory mutation, a deletion, an insertion, an inversion, a splice mutation and a duplication.

72. The isolated differentiated cell, tissue or organ of claim 69, wherein said disease-causing mutation is associated with a genetic disorder selected from the group consisting of cystic fibrosis (CF), myotonic dystrophy (DM), van Waardenburg syndrome (WS), metachromatic leukodystrophy (MLD), Gorlin disease, Huntington's disease (HD), spinal muscular atrophy (SMA) and Duchenne muscular dystrophy (DMD).

73. The isolated differentiated cell, tissue or organ of claim 69, wherein said disease-causing mutation is selected from the group consisting of the W1282X as set forth in SEQ ID NO:24 associated with cystic fibrosis, the PAX3-del28 (510del28 in SEQ ID NO:34) associated with van Waardenburg syndrome, more than 50 (CTG) repeats as set forth in SEQ ID NO:22 associated with Myotonic dystrophy and the 1505C→T (P377L) as set forth in SEQ ID NO:21 associated with metachromatic leukodystrophy.

74. A method of identifying an agent suitable for treating a disorder associated with at least one disease-causing mutation, comprising:
(a) generating a stem cell line or an embryoid body carrying the at least one disease-causing mutation;
(b) subjecting cells of said stem cell line or said embryoid body to differentiating conditions to thereby obtain differentiated cells exhibiting an effect of the at least one disease-causing mutation and;
(c) exposing said differentiated cells to a plurality of molecules and identifying from said plurality of molecules at least one molecule capable of regulating said effect of the at least one disease-causing mutation on said differentiated cells, said at least one molecule being the agent suitable for treating the disorder associated with the at least one disease-causing-mutation.

75. The method of claim 74, wherein said embryoid body is derived from a stem cell or a stem cell line.

76. The method of claim 74, wherein said stem cell is of embryonic origin.

77. The method of claim 74, wherein said stem cell is of human origin.

78. The method of claim 74, wherein said stem cell exhibits a karyotype of 46, XX or 46, XY following at least 30 passages.

79. The method of claim 74, wherein said disease-causing mutation is selected from the group consisting of a missense mutation, a nonsense mutation, a frameshift mutation, a readthrough mutation, a promoter mutation, a regulatory mutation, a deletion, an insertion, an inversion, a splice mutation and a duplication.

80. The method of claim 74, wherein said disease-causing mutation is associated with a genetic disorder selected from the group consisting of cystic fibrosis (CF), myotonic dystrophy (DM), van Waardenburg syndrome (WS), metachromatic leukodystrophy (MLD), Gorlin disease, Huntington's disease (HD), spinal muscular atrophy (SMA) and Duchenne muscular dystrophy (DMD).

81. The method of claim 74, wherein said disease-causing mutation is selected from the group consisting of the W1282X as set forth in SEQ ID NO:24 associated with cystic fibrosis, the PAX3-del28 (510del28 in SEQ ID NO:34) associated with van Waardenburg syndrome, more than 50 (CTG) repeats as set forth in SEQ ID NO:22 associated with Myotonic dystrophy and the 1505C→T (P377L) as set forth in SEQ ID NO:21 associated with metachromatic leukodystrophy.

82. The method of claim 74, further comprising a step of isolating lineage specific cells from said embryoid body prior to step (b).

83. The method of claim 82, wherein said isolating lineage specific cells is effected by sorting of cells contained within said embryoid body via fluorescence activated cell sorter.

84. The method of claim 82, wherein said isolating lineage specific cells is effected by a mechanical separation of cells, tissues and/or tissue-like structures contained within said embryoid body.


SEQUENCE LISTING (38 sequences)

SEQ ID NO: 1 - DNA, 26 bp, Artificial sequence
  Single strand DNA oligonucleotide
cttcccaggcctgcagtttgcccatc

SEQ ID NO: 2 - DNA, 27 bp, Artificial sequence
  Single strand DNA oligonucleotide
gaacggggctcgaagggtccttgtagc

SEQ ID NO: 3 - DNA, 22 bp, Artificial sequence
  Single strand DNA oligonucleotide
gaagggtccttgtagccgggaa

SEQ ID NO: 4 - DNA, 23 bp, Artificial sequence
  Single strand DNA oligonucleotide
gggatcacagaccatttctttct

SEQ ID NO: 5 - DNA, 20 bp, Artificial sequence
  Single strand DNA oligonucleotide
cttcccacagtgtccactcc

SEQ ID NO: 6 - DNA, 20 bp, Artificial sequence
  Single strand DNA oligonucleotide
gaggattgcaaggcttatgg

SEQ ID NO: 7 - DNA, 20 bp, Artificial sequence
  Single strand DNA oligonucleotide
acggcaggccgctgcccaac

SEQ ID NO: 8 - DNA, 17 bp, Artificial sequence
  Single strand DNA oligonucleotide
agtctgggagccaggag

SEQ ID NO: 9 - DNA, 23 bp, Artificial sequence
  Single strand DNA oligonucleotide
gagaacaatgagaaccttcagga

SEQ ID NO: 10 - DNA, 23 bp, Artificial sequence
  Single strand DNA oligonucleotide
ttctggcgccggttacagaacca

SEQ ID NO: 11 - DNA, 24 bp, Artificial sequence
  Single strand DNA oligonucleotide
tgcttgaatgtgctgatgacaggg

SEQ ID NO: 12 - DNA, 24 bp, Artificial sequence
  Single strand DNA oligonucleotide
aaggcaagtcagcagccatctcat

SEQ ID NO: 13 - DNA, 25 bp, Artificial sequence
  Single strand DNA oligonucleotide
gctggattgtctgcaggatggggaa

SEQ ID NO: 14 - DNA, 25 bp, Artificial sequence
  Single strand DNA oligonucleotide
tcccctgaagaaaattggttaaaat

SEQ ID NO: 15 - DNA, 22 bp, Artificial sequence
  Single strand DNA oligonucleotide
gagtgaaatggcacgataccta

SEQ ID NO: 16 - DNA, 22 bp, Artificial sequence
  Single strand DNA oligonucleotide
tttcctctccttcttcaccttc

SEQ ID NO: 17 - DNA, 22 bp, Artificial sequence
  Single strand DNA oligonucleotide
ggagttatggtgggtatgggtc

SEQ ID NO: 18 - DNA, 22 bp, Artificial sequence
  Single strand DNA oligonucleotide
agtggtgacaaaggagtagcca

SEQ ID NO: 19 - DNA, 32 bp, Artificial sequence
  Single strand DNA oligonucleotide
atctggcaccacaccttctacaatgagctgcg

SEQ ID NO: 20 - DNA, 32 bp, Artificial sequence
  Single strand DNA oligonucleotide
cgtcatactcctgcttgctgatccacatctgc

SEQ ID NO: 21 - DNA, 2039 bp, Homo sapiens
  misc_feature (1505)..(1505): Harboring a C to T mutation in the P377L mutant
cggaagcgcccgcagcccggtaccggctcctcctgggctccctctagcgccttccccccggcccgactccgctggtcagcgccaagtgacttacgcccccgaccctgagcccggaccgctaggcgaggaggatcagatctccgctcgagaatctgaaggtgccctggtcctggaggagttccgtcccagcccgcggtctcccggtactgtcgggccccggccctctggagcttcaggaggcggccgtcagggtcggggagtatttgggtccggggtctcagggaagggcggcgcctgggtctgcggtatcggaaagagcctgctggagccaagtagccctccctctcttgggacagacccctcggtcccatgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctaccngtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctgagggcccctcggctggcctgggcatgtgatggctcctcactgggagcctgtgggggaggctcaggtgtctggagggggtttgtgcctgataacgtaataacaccagtggagacttgcacatctgaaaaaaaaaaaaaaaaa

SEQ ID NO: 22 - DNA, 2892 bp, Homo sapiens
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaaaaaaaaaaaaaaaaaaaaaaaaaaaa

SEQ ID NO: 23 - DNA, 1489 bp, Homo sapiens
aggaggagactcaggcaggccgcgctccagcctcaccaggctccccggctcgccgtggctctctgagcccccttttcagggaccccagtcgctggaacatttgcccagactcgtaccaaacttttccgccctgggctcgggatcctggactccggggcctccccgtcctcccctttcccgggttccagctccggcctctggactaggaaccgacagcccccctccccgcgtccctccctctctctccagccgttttggggaggggctctccacgctccggatagttcccgagggtcatccgcgccgcactcgcctttccgtttcgccttcacctggatataatttccgagcgaagctgcccccaggatgaccacgctggccggcgctgtgcccaggatgatgcggccgggcccggggcagaactacccgcgtagcgggttcccgctggaagtgtccactcccctcggccagggccgcgtcaaccagctcggcggcgtttttatcaacggcaggccgctgcccaaccacatccgccacaagatcgtggagatggcccaccacggcatccggccctgcgtcatctcgcgccagctgcgcgtgtcccacggctgcgtctccaagatcctgtgcaggtaccaggagactggctccatacgtcctggtgccatcggcggcagcaagcccaagcaggtgacaacgcctgacgtggagaagaaaattgaggaatacaaaagagagaacccgggcatgttcagctgggaaatccgagacaaattactcaaggacgcggtctgtgatcgaaacaccgtgccgtcagtgagttccatcagccgcatcctgagaagtaaattcgggaaaggtgaagaggaggaggccgacttggagaggaaggaggcagaggaaagcgagaagaaggccaaacacagcatcgacggcatcctgagcgagcgaggtaagcggtggcgccttgggcggcgcacttgctgggtgacttggagggcatcggctagctgacattggtgatctgacggcagccaagcccagctcggatcaaggtcccttcatgcgcggtgtctctgcgcctgagtaacgacatggaactgaaagaccagagggacactaggaatcaaaacaaacatttctattctgcttagtttttctgttttgtaaatctttctttcttaaccactttcagcccctgggattctagaactgtgaattgtgctctgttgtagggggcaggggaagctctcactctgttgccattaaatgtatgagactgggcatctctgagcaattgtagggccggggatagagggtacttgaatcttcagaagttgaagtagcttttatgccctcaggaaaggccctggtctccggagtttcctcgcattaaaggagagagagagagagtactcttttgggcaacggccctccaaaattgcccccacattggctgccttataaatatgtctgtgtgttgactggt

SEQ ID NO: 24 - DNA, 6129 bp, Homo sapiens
  misc_feature (1609)..(1609): harboring the C to T mutation causing cystic fibrosis
aattggaagcaaatgacatcacagcaggtcagagaaaaagggttgagcggcaggcacccagagtagtaggtctttggcattaggagcttgagcccagacggccctagcagggaccccagcgcccgagagaccatgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatgatgattatgggagaactggagccttcagagggtaaaattaagcacagtggaagaatttcattctgttctnagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgaatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcctttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttagagagcagcataaatgttgacatgggacatttgctcatggaattggagctcgtgggacagtcacctcatggaattggagctcgtggaacagttacctctgcctcagaaaacaaggatgaattaagtttttttttaaaaaagaaacatttggtaaggggaattgaggacactgatatgggtcttgataaatggcttcctggcaatagtcaaattgtgtgaaaggtacttcaaatccttgaagatttaccacttgtgttttgcaagccagattttcctgaaaacccttgccatgtgctagtaattggaaaggcagctctaaatgtcaatcagcctagttgatcagcttattgtctagtgaaactcgttaatttgtagtgttggagaagaactgaaatcatacttcttagggttatgattaagtaatgataactggaaacttcagcggtttatataagcttgtattcctttttctctcctctccccatgatgtttagaaacacaactatattgtttgctaagcattccaactatctcatttccaagcaagtattagaataccacaggaaccacaagactgcacatcaaaatatgccccattcaacatctagtgagcagtcaggaaagagaacttccagatcctggaaatcagggttagtattgtccaggtctaccaaaaatctcaatatttcagataatcacaatacatcccttacctgggaaagggctgttataatctttcacaggggacaggatggttcccttgatgaagaagttgatatgccttttcccaactccagaaagtgacaagctcacagacctttgaactagagtttagctggaaaagtatgttagtgcaaattgtcacaggacagcccttctttccacagaagctccaggtagagggtgtgtaagtagataggccatgggcactgtgggtagacacacatgaagtccaagcatttagatgtataggttgatggtggtatgttttcaggctagatgtatgtacttcatgctgtctacactaagagagaatgagagacacactgaagaagcaccaatcatgaattagttttatatgcttctgttttataattttgtgaagcaaaattttttctctaggaaatatttattttaataatgtttcaaacatatattacaatgctgtattttaaaagaatgattatgaattacatttgtataaaataatttttatatttgaaatattgactttttatggcactagtatttttatgaaatattatgttaaaactgggacaggggagaacctagggtgatattaaccaggggccatgaatcaccttttggtctggagggaagccttggggctgatcgagttgttgcccacagctgtatgattcccagccagacacagcctcttagatgcagttctgaagaagatggtaccaccagtctgactgtttccatcaagggtacactgccttctcaactccaaactgactcttaagaagactgcattatatttattactgtaagaaaatatcacttgtcaataaaatccatacatttgtgt

SEQ ID NO: 25 - DNA, 22 bp, Artificial sequence
  Single strand DNA oligonucleotide
gcaccattaaagaaaatatgat

SEQ ID NO: 26 - DNA, 19 bp, Artificial sequence
  Single strand DNA oligonucleotide
ctcttctagttggcatgct

SEQ ID NO: 27 - DNA, 20 bp, Artificial sequence
  Single strand DNA oligonucleotide
taatggatcatgggccatgt

SEQ ID NO: 28 - DNA, 20 bp, Artificial sequence
  Single strand DNA oligonucleotide
acagtgttgaatgtggtgca

SEQ ID NO: 29 - DNA, 16 bp, Artificial sequence
  Single strand DNA oligonucleotide
gttgttggaggttgct

SEQ ID NO: 30 - DNA, 16 bp, Artificial sequence
  Single strand DNA oligonucleotide
gttgttggcggttgct

SEQ ID NO: 31 - DNA, 20 bp, Artificial sequence
  Single strand DNA oligonucleotide
gcagagtacctgaaacagga

SEQ ID NO: 32 - DNA, 20 bp, Artificial sequence
  Single strand DNA oligonucleotide
ggcataatccaggaaaactg

SEQ ID NO: 33 - DNA, 20 bp, Artificial sequence
  Single strand DNA oligonucleotide
ggcataatccaggaaaacta

SEQ ID NO: 34 - DNA, 601 bp, Homo sapiens
ctgcagggtctcctaagttgccactccccgggcccgaaggaagacgctttctctggggagaattattttgccccatttgctgtctttacacaagattacatgcgctatattgctgaataatttgaacaaatgtgattgaaaggaggtctgggtctgggagtccgatgtcgagcagtttcagcgtcggtgctgtaacatgaggataggccagaagactgcgaaattacgtgctgctgttctttgctttttattttcctccagtgacttttcccttgcttctctttttcaccttcccacagtgtccactcccctcggccagggccgcgtcaaccagctcggcggcgtttttatcaacggcaggccgctgcccaaccacatccgccacaagatcgtggagatggcccaccacggcatccggccctgcgtcatctcgcgccagctgcgcgtgtcccacggctgcgtctccaagatcctgtgcaggtaccaggagactggctccatacgtcctggtgccatcggcggcagcaagcccaaggtgagcgggcgggccttgccctcctggctcccagactgtggtcccctgtgttggggaagaccgggc

SEQ ID NO: 35 - DNA, 20 bp, Artificial sequence
  Single strand DNA oligonucleotide
tacctatatgtcacagaagt

SEQ ID NO: 36 - DNA, 20 bp, Artificial sequence
  Single strand DNA oligonucleotide
gtacaagtatcaaatagcag

SEQ ID NO: 37 - DNA, 20 bp, Artificial sequence
  Single strand DNA oligonucleotide
gcagtctctcttcttctagc

SEQ ID NO: 38 - DNA, 20 bp, Artificial sequence
  Single strand DNA oligonucleotide
aggggccagggatctagggc